(19) United States
(12) Patent Application Publication
(10) Pub. No.: US 2012/0231069 A1
(43) Pub. Date: Sep. 13, 2012

(54) Targeted Nanocarrier Systems for Delivery of Actives Across Biological Membranes

(75) Inventors: David P. Nowotnik, Dallas, TX (US); Ryszard Zarzycki, Dallas, TX (US); Paul Sood, Dallas, TX (US); N.Rao Ummaneni, Dallas, TX (US)

(73) Assignee: Access Pharmaceuticals, Inc.

(21) Appl. No.: 13/414,662

(22) Filed: Mar. 7, 2012

Related U.S. Application Data
(60) Provisional application No. 61/450,541, filed on Mar. 8, 2011.

(51) Int. Cl. A61K 9/127; A61K 9/51; B82Y 5/00

(52) U.S. Cl. 424/450; 424/490; 977/773; 977/906; 977/907

(57) ABSTRACT

Disclosed herein are nanoparticle, micelle and/or liposome compositions, each comprising a therapeutic agent encapsulated in one or more polymer(s), wherein a vitamin B12 or a derivative thereof is attached to the one or more polymer(s) via a linker group, as well as methods for making and using same.

### CROSS-REFERENCE TO RELATED APPLICATIONS

This application claims priority under 35 U.S.C. §119(e) of U.S. Provisional Application No. 61/450,541, filed Mar. 8, 2011, the contents of which are incorporated by reference in its entirety.

### BACKGROUND

While treatment of disease using pharmaceutically-active compounds is commonplace, the development of medications is challenged by the need to deliver the drug conveniently to the sites of action in sufficient quantities to achieve the desired pharmacological effect. A convenient route of drug administration is oral delivery. It may be preferred by patients as it is non-invasive and by physicians for patient compliance. Yet this route may be inaccessible for many pharmaceutically-active compounds either because these compounds are broken down in the gastrointestinal tract or fail to be absorbed. Similarly, drugs that may show significant promise in early testing can fail because the compounds may not reach their intended sites of action for failure to cross biological membranes. For example, in cancer chemotherapy, it may often be necessary to dose patients with high levels of cytotoxic drugs in order to achieve a meaningful therapeutic effect which may also result in damage to normal cells, resulting in significant adverse side-effects.

It may be desirable to alter the natural biodistribution of cytotoxic compounds so that more of the drug is delivered to tumor cells, and less to normal tissues. Monoclonal antibodies to tumor-specific antigens have been used as target cytotoxic agents to tumors so as to improve upon the therapeutic index (a ratio of a drug's beneficial effects compared with its adverse side-effects). The use of monoclonal antibodies, however, may generate other issues, such as immunogenicity, whereby the patient's immune systems may develop an immune response to the antibody-drug conjugate.

It is therefore desirable to have new drug delivery systems that are both safe and effective, and which can facilitate the delivery of drugs across biological membrane such as the gut wall (in oral drug delivery) and cell membranes (in the treatment of disease). This invention satisfies this need and provides related advantages as well.

### SUMMARY OF THE INVENTION

The invention relates to the delivery of pharmaceutically-active compounds such as small-molecule drugs, proteins, peptides and oligonucleotides across biological barriers using naturally-occurring vitamin transport systems. In one aspect, the invention relates to the delivery of pharmaceutically-active compounds utilizing vitamin B12 transport systems with the protection of the pharmaceutically-active compound during transport by incorporation of the compound in nanocarriers, such as, but not limited to, nanostructures containing surface-linked vitamin B12 or a derivative thereof. In some embodiments, the nanocarriers are made from synthetic, semi-synthetic polymers or naturally-occurring polymers. In other embodiments, the nanocarriers are made by polymer-coating nanoparticulate cores comprising the active optionally mixed with polymers and/or other pharmaceutically-acceptable excipients. In other embodiments, the nanocarriers are liposomes or micelles made from hydrophobic molecules with hydrophilic end groups.

The invention also relates to processes for preparing the nanocarriers, pharmaceutical compositions containing same and methods of drug delivery and treatment of disease involving the nanostructures.

Surprisingly it has been found by the Applicants that nanocarriers capable of drug delivery can be formed by incorporating the small, hydrophilic vitamin B12 molecule or a derivative thereof as the primary targeting group and optionally other physically-bound or covalently-linked molecules for targeting or delivery. The nanoparticle systems of this invention can be used for drug delivery of pharmaceutically-active compounds entrapped within the nanocarrier and/or of pharmaceutically active compounds bound to one component of the carrier system. Drug delivery by nanocarrier systems of this invention can be either oral drug delivery, whereby the nanocarriers are transferred from the intestinal lumen into the bloodstream, and/or through targeting of nanocarriers in the bloodstream to diseased cells in the body that over express the receptors that facilitate the cell uptake of vitamin B12 and/or the other nanocarrier-attached targeting groups.

Thus, in one embodiment, the present disclosure provides a nanoparticle comprising, or alternatively consisting essentially of, or yet alternatively consisting, a therapeutic agent encapsulated by one or more polymer(s) and vitamin B12 or a derivative thereof attached to the at least one polymer via a linker group. In one aspect, the nanoparticle further comprises a targeting agent other than vitamin B12 attached to the at least one polymer.

In another embodiment, the present disclosure provides a micelle comprising, or alternatively consisting essentially of, or yet alternatively consisting, a therapeutic agent encapsulated by the micelle and vitamin B12 or a derivative thereof attached to the micelle as a targeting agent. In one aspect, the micelle further comprises a second targeting agent other than vitamin B12 attached to the micelle.

Also provided is a liposome comprising, or alternatively consisting essentially of, or yet alternatively consisting, the micelle of any of the above embodiments. Further provided, in one embodiment, is a liposome comprising, or alternatively consisting essentially of, or yet alternatively consisting, a therapeutic agent encapsulated by the liposome and vitamin B12 or a derivative thereof attached to the liposome as a targeting agent.

In some aspects, the liposome further comprises a second targeting agent other than vitamin B12 or the derivative attached to the liposome.

In one aspect of the above embodiment, the vitamin B12 or a derivative thereof is attached to the at least on polymer on the surface of the nanoparticle and/or embedded within the nanoparticle or micelle. The vitamin B12 or a derivative thereof is attached to the one or more polymer(s) covalently or physically. Non-limiting examples of the physical attachment comprises an electrostatic binding interaction between charged groups on the VB12 derivative and oppositely-charged regions of the nanoparticle or micelle, or a hydrophobic binding interaction between hydrophobic groups on the VB12 derivative and hydrophobic regions of the nanoparticle or micelle. Non-limiting examples of VB12 derivatives include VB12-5′-O-carboxytriazole, VB12-5′-O-carboxylmidazole, VB12-5′-O-carboxyamido-C2-C20-alkylamines, VB12-5′-O-carboxyamido-oligoethyleneoxyamines, and dicarboxylic acid derivatives of the aforementioned compounds.

In another aspect of the above embodiments, the one or more polymer(s) comprise a degradable polymer or a stable polymer(s), e.g., one or more of dextran, carboxymethyl dextran, chitosan, trimethylchitosan or poly(lactic-co-glycolic acid) (PLGA), polylactic acid (PLA), polyglycolic acid (PGA), polyvinylalcohol (PVA), polyanhydrides, polyacylates, polymethacrylates, polyacylamides, polymethacrylate, dextran, chitosan, cellulose, hypromellose, starch, dendrimers, peptides, proteins, polyethyleneglycols and poly(ethyleneglycol-co-propyleneglycol), and synthetic derivatives of the aforementioned polymers.

In another aspect of the above embodiments, the linkers attaching the VB12 or a derivative is the same or different and is selected from the group of a short peptide chain (H-[NHCHR—CO]n—OH) where n is 1-20 and R is the same or different for each of the n amino acids, and is one of the 22 side groups known to be present in natural amino acids; a short alkyl chain (CH₂)ₙ where n=2-10, terminated by two amino groups or two carboxyl groups or one amino group and one carboxyl group; an oligoethyleneoxy chain (CH₂CH₂O)ₙ where n=2-100, terminated by two amino groups or two carboxyl groups or one amino group and one carboxyl group; a poly(lactic-co-glycolic acid) (PLGA), polylactic acid (PLA), polyglycolic acid (PGA) chain of average molecular weight of 2 kDa to 70 kDa terminated by two amino groups or two carboxyl groups or one amino group and one carboxyl group; and any combination of two or more of any of the aforementioned linkers.

Non-limiting examples of a therapeutic agent is selected from the group consisting of a small or large synthetic molecule, protein, peptide, glycoprotein, nucleoside, nucleotide, humanized monoclonal antibody, non-humanized monoclonal antibody, therapeutically relevant fragments of humanized and/or non-humanized monoclonal antibody, and agents for effecting RNA interference (RNAi) comprising dsRNA, siRNA, miRNA or antisense RNA, or the combinations thereof. In some aspects, the therapeutic agent is selected from the group consisting of analgesic, antiallergenic, antianginal agent, antiarrythmic drug, antibiotic, anticoagulant, antidementia drug, antidepressant, antidiabetic, antihistamine, antihypertensive, anti-inflammatory, antineoplastic agent, antiparasitic, antipyretic, antiretroviral drug, antiulcerative agent, antiviral agent, cardiovascular drug, cholesterol-lowering agent, CNS active drug, a hormone, growth hormone inhibitor, growth hormone, hematopoietic drug, hemostatic, hypotensive diuretic, keratolytic, therapeutic for osteoporosis, vaccine, vasoconstrictor, and vasodilator.

Compositions are also provided. In one embodiment, the composition comprises, or alternatively consists essentially of, or yet alternatively consists of, a carrier and one or more of the nanoparticle, the micelle or the liposome of the above embodiments. In one aspect, the carrier is a pharmaceutically acceptable carrier. In another aspect, the composition is formulated for oral administration.

Yet in another embodiment, the present disclosure provides a method for delivering a therapeutic agent in vivo, comprising administering to a subject an effective amount of the nanoparticle, the micelle, the liposome or the composition of any of the above embodiments, thereby delivering the therapeutic agent. Yet provided is use of nanoparticle, the micelle, the liposome or the composition of any of the above embodiments, in the preparation of a medicament.

In a further embodiments, provided is a method for preparing a nanoparticle composition comprising, or alternatively consisting essentially of, or yet alternatively consisting, admixing a therapeutic agent and at least one polymer to which vitamin B12 or a derivative thereof is attached by a linker group in a suitable solvent and optionally, wherein the ratio of the polymer to the therapeutic agent is in a range selected from the group of 1 to 15%, 1 to 40%, 5 to 50%, 5 to 40%, 5 to 30%, 10 to 35%, or 10 to 30%, or the combinations thereof.

In one aspect, the method further comprises admixing a second targeting agent other than vitamin B12 or the derivative in the suitable solvent. In another aspect, the method further comprises linking to the at least one polymer a second targeting agent other than vitamin B12. In yet another aspect, the method further comprises modifying the nanoparticles to effect cross-linking of components of the nanoparticles wherein the components comprise metal ions, small molecules having at least two positively charged groups or two negatively-charged groups, or small molecules that react to form at least two covalent bonds. In yet another aspect, the solvent is >50% water.

In another aspect, the method further comprises isolating, purifying, and/or drying resultant nanoparticles from the solvent. In one aspect, the nanoparticles are isolated by solvent evaporation. In another aspect, the nanoparticles are isolated by dialysis or tangential flow filtration. In another aspect, the nanoparticles are isolated by filtration or centrifugation. In yet another aspect, nanoparticles are isolated by addition of a cosolvent followed by filtration or centrifugation.

Yet in another aspect, the method further comprises purifying the nanoparticles by washing the nanoparticles with a suitable solvent.

### BRIEF DESCRIPTION OF THE FIGURES

FIG. 1 depicts a structure of vitamin B12 in which R represents a monodentate axial ligand as defined later. In either of the above manifestations of the present invention, attachment of vitamin B12 can occur directly to one of the above mentioned components of the nanoparticle or via a suitable linker. Vitamin B12 attachment can occur via either the 2′ or 5′-oxygen atoms on the ribose unit of vitamin B12 (as exemplified by U.S. Pat. No. 6,150,341), or via conversion of one or more of the amide groups to carboxyl and subsequent addition of a linker group (see, for example, Waibel et al, Cancer Res., 2008, 68, 2904-2911) or by replacement of the axial ligand (R) on the cobalt atom of vitamin B12 with a bifunction ligand or a compound that can bind to cobalt, as exemplified in U.S. Pat. No. 6,262,253.

FIG. 2 depicts three exemplary nanoparticle constructs of this invention, termed nanocapsule, polymer nanoparticle, and drug nanoparticle. In the nanocapsule, a small nanoparticle of the drug or drug formulated with polymers and/or other pharmaceutically-acceptable excipients (shown in white at the center of the nanoparticle) is coated by either a natural or synthetic polymer or a lipid (in the case of a micelle or liposome). Vitamin B12 (depicted as black circles) and optionally other targeting groups (grey and white circles) are bound to the surface of the nanoparticle either covalently or physically through an optional linker group. In the case of a polymer nanoparticle, the drug, polymers and optional pharmaceutically-acceptable excipients which form the nanoparticle are intimately mixed. The vitamin B12 and optional other targeting groups can be bound to the polymers prior to nanoparticle formation or they can be attached after nanoparticle formation. For nanoparticle formation involving polymers to which targeting groups are attached, some targeting groups will be embedded in the nanoparticle and some will be presented on the surface of the nanoparticle. In the case of drug nanoparticle, a larger nanoparticle of drug or mixture of drug and other excipients is coated with thin layer of polymers optionally containing other excipients. The targeting groups can either be attached to the polymers prior to coating or linked to the polymer after coating of the drug nanoparticle.

FIG. 3 is plot showing inhibition of tumor growth with Abraxane or Cobrazane Conjugates. Athymic nude mice were implanted with human leukemia K562 cells and xenograft tumors allowed to grow until 150-200 mm³ in size. Animals were randomized into groups of seven and dosed by intraperitoneal injection with either saline control, Abraxane (200 mg/kg paclitaxel) or Cobraxane (100 or 200 mg/kg paclitaxel) and tumor sizes were measured three times per week. The plot shows inhibition of tumor growth (relative to saline control) for all three active groups. Cobraxane at 50% of paclitaxel dose was superior to Abraxane and an equivalent dose of Cobraxane actually reduced the tumor size.

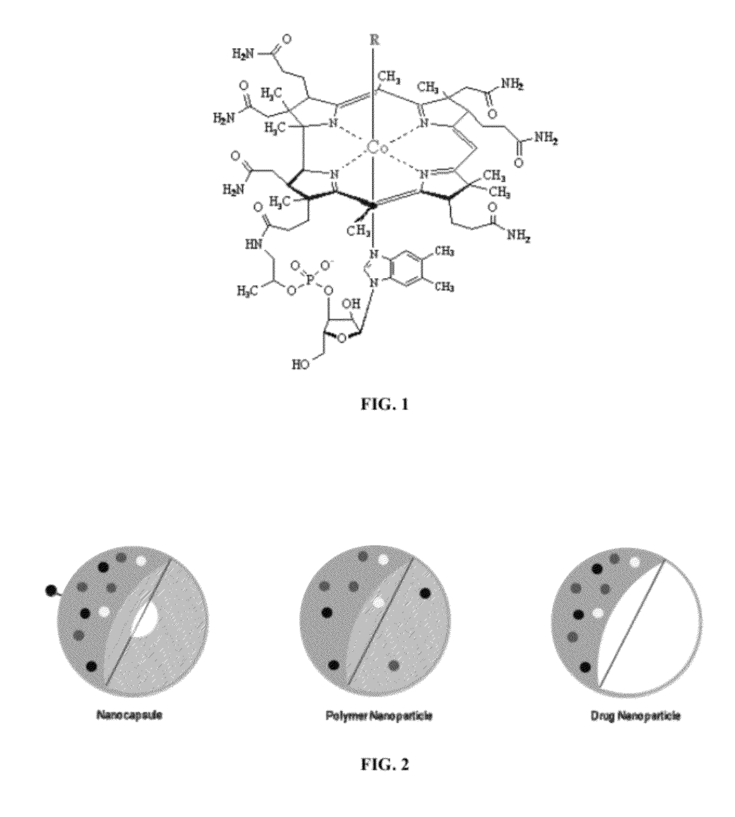

### DETAILED DESCRIPTION OF THE INVENTION

All technical and patent publications cited herein are incorporated herein by reference in their entirety.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise.

“Comprising” refers to compounds, compositions and methods including the recited elements, but not exclude others. “Consisting essentially of,” when used to define compounds, compositions or methods, shall mean excluding other elements that would materially affect the basic and novel characteristics of the claimed technology. “Consisting of,” shall mean excluding any element, step, or ingredient not specified in the claim. Embodiments defined by each of these transition terms are within the scope of this technology.

As used herein, “nanoparticle”, “nanocarrier”, or “nanostructure” refers a microscopic particle less than about 1 micron in diameter. In some embodiments, the nanoparticles range in size from about 1 nm to about 1,000 nm diameter, or alternatively between about 10 nm to about 1000 nm, or alternatively between about 10 nm to about 900 nm, or alternatively between about 10 nm to about 800 nm, or alternatively between about 10 nm to about 700 nm, or alternatively between about 10 nm to about 600 nm, or alternatively between about 10 nm to about 500 nm, or alternatively between about 20 nm to about 1000, or alternatively between about 20 nm to about 800 nm, or alternatively between about 20 nm to about 700 nm, or alternatively between about 20 nm to about 600 nm, or alternatively between about 20 nm to about 500 nm; or alternatively between about 30 nm to about 1000 nm, or alternatively between about 30 nm to about 900 nm, or alternatively between about 30 nm to about 800 nm, or alternatively between about 30 nm to about 700 nm, or alternatively between about 100 nm to about 900 nm, or alternatively between about 200 nm to about 1000 nm, or alternatively between about 300 nm to about 1000 nm, or alternatively between about 400 nm to about 1000 nm, or alternatively between about 500 nm to about 1000 nm; or alternatively between about 600 nm to about 1000 nm; or alternatively between about 700 nm to about 1000 nm; or alternatively between about 800 nm to about 1000 nm; or alternatively between about 900 nm to about 1000 nm; or alternatively between about 100 nm to about 300 nm; or alternatively between about 200 nm to about 600 nm; or alternatively between about 300 nm to about 600 nm; or alternatively between about 500 nm to about 800 nm.

As used herein, “polymer” refers to a naturally-occurring, synthetic or semi-synthetic large molecule (macromolecule) typically composed of repeating structural units connected by covalent chemical bonds. Polymers useful for the implementation of this invention have molecular weights in the range of 1 to 5000 kDa. The polymers can be stable, degradable and made of random copolymers or block copolymers.

As used herein, “random copolymer” refers to a polymer comprising two or more repeating structural units in which the sequence of the individual repeating structural units is random and not predetermined or defined.

As used herein, “block copolymer” refers to a polymer comprising two or more repeating structural units in which individual repeating structural units are connected to each other forming identifiable blocks of repeating structural units within the complete polymer strand.

As used herein, “charged group” refers to a chemical functional group that is fully ionized resulting in that group having either a positive or a negative charge, or possibly multiple positive or multiple negative charges. Polymers could have multiple charged groups either as components of the polymer chain, and/or as attachments to the polymer, either direct attachment or by way of a linker. Polymer charged groups may be either naturally-occurring or synthetic. A charged group may be part of a therapeutically active compound, either as an intrinsic component of that compound or as a synthetic analog of the therapeutically active compound, for example a prodrug.

As used herein, “ionisable group” refers to a chemical functional group that is partially ionized at or close to physiological pH resulting in that group having either a partial positive or a partial negative charge. The charge of an ionisable group will vary with pH. Polymers could have multiple ionisable groups either as components of the polymer chain, and/or as attachments to the polymer, either direct attachment or by way of a linker. Polymer ionisable groups may be either naturally-occurring or synthetic. A ionisable group may be part of a therapeutically active compound, either as an intrinsic component of that compound or as a synthetic analog of the therapeutically active compound, for example a prodrug.

As used herein, “polyelectrolyte complex” or “PEC” refers to a three-dimensional structure resulting from the formation of multiple ionic bonds between two or more compounds having chemical functional groups that are charged and/or ionisable wherein at least one compound possesses a net negative charge and at least one compound has a net positive charge, and at least one compound preferentially is a polymer. The diameter of PECs can typically range from 1 nm to several microns, with average particle size and particle size distribution controlled by the chemical and physical nature of the constituent components and method of preparation. PECs can be water soluble (i.e. suspension of nanoparticles in water results in a clear, transparent liquid) or insoluble (i.e. suspension of nanoparticles in water results in a cloudy liquid). PEC nanoparticles typically can range in size from about 1 nm to about 1,000 nm diameter, or alternatively about 5 nm to about 400 nm or alternatively about 10 nm to about 300 nm.

As used herein, the term “polynucleotides” includes deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and “thymidine” are used. It is understood that if the nucleic acid is RNA, a nucleotide having a uracil base is uridine.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching. The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles.

As used herein, the term “carrier” encompasses any of the standard carriers, such as a phosphate buffered saline solution, buffers, water, and emulsions, such as an oil/water or water/oil emulsion, and various types of wetting agents. The compositions also can include stabilizers and preservatives. In one aspect of the invention, the carrier is a buffered solution such as, but not limited to, a PCR buffer solution.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection, sometimes called transduction), transfection, transformation or various other protein-based or lipid-based gene delivery complexes) as well as techniques facilitating the delivery of “naked” polynucleotides (such as electroporation, “gene gun” delivery and various other techniques used for the introduction of polynucleotides). Unless otherwise specified, the term transfected, transduced or transformed may be used interchangeably herein to indicate the presence of exogenous polynucleotides or the expressed polypeptide therefrom in a cell. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

“RNA interference” (RNAi) refers to sequence-specific or gene specific suppression of gene expression (protein synthesis) that is mediated by short interfering RNA (siRNA).

“Short interfering RNA” (siRNA) refers to double-stranded RNA molecules (d5RNA), generally, from about 10 to about 30 nucleotides in length that are capable of mediating RNA interference (RNAi), or 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, or 29 nucleotides in length. As used herein, the term siRNA includes short hairpin RNAs (shRNAs). A siRNA directed to a gene or the mRNA of a gene may be a siRNA that recognizes the mRNA of the gene and directs a RNA-induced silencing complex (RISC) to the mRNA, leading to degradation of the mRNA. A siRNA directed to a gene or the mRNA of a gene may also be a siRNA that recognizes the mRNA and inhibits translation of the mRNA.

“Double stranded RNA” (dsRNA) refer to double stranded RNA molecules that may be of any length and may be cleaved intracellularly into smaller RNA molecules, such as siRNA. In cells that have a competent interferon response, longer dsRNA, such as those longer than about 30 base pair in length, may trigger the interferon response. In other cells that do not have a competent interferon response, dsRNA may be used to trigger specific RNAi.

microRNA or miRNA are single-stranded RNA molecules of 21-23 nucleotides in length, which regulate gene expression. miRNAs are encoded by genes from whose DNA they are transcribed but miRNAs are not translated into protein (non-coding RNA); instead each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

A siRNA vector, dsRNA vector or miRNA vector as used herein, refers to a plasmid or viral vector comprising a promoter regulating expression of the RNA. “siRNA promoters” or promoters that regulate expression of siRNA, dsRNA, or miRNA are known in the art, e.g., a U6 promoter as described in Miyagishi and Taira (2002) Nature Biotech. 20:497-500, and a H1 promoter as described in Brummelkamp et al. (2002) Science 296:550-3.

As used herein, “degradable polymer” refers to a polymer which can be broken down under specific conditions to smaller units In one aspect, repeated degradation of the polymer units in situ (in the body) allows for small fragments to be excreted or otherwise eliminated.

As used herein, “stable polymer” refers to a polymer in which the main structure (backbone) of the polymer cannot be broken under conditions typically found in the body. In a stable polymer, it remains possible that functional groups attached to the polymer backbone can be modified or degraded under conditions typically found in the body.

As used herein, “alkyl” refers to a saturated (containing no multiple carbon-carbon bonds) aliphatic (containing no delocalized it-electron system), hydrocarbon containing, if otherwise unsubstituted, only carbon and hydrogen atoms. The designation (n1C-n2C)alkyl, wherein n1 and n2 are integers from one to 6, refers to straight or branched chain alkyl groups comprising from n1 to and including n2 carbon atoms. An alkyl group herein may be optionally substituted with one or more entities selected from the group consisting of halo, hydroxy, alkoxy, aryloxy, carbonyl, nitro, cyano, carboxyl and alkoxycarbonyl.

As used herein, “linker” refers to a group of atoms that is used to couple a polymeric backbone to another function or group to spatially separate the two entities. Thus, a linker of this invention has an essentially longitudinal axis, that is, it is essentially linear rather than highly branched or clumped, although the structure will, of course, not be exactly linear due to the angular constraints placed on the structure by required bond angles between covalently bonded atoms. Examples of linkers include, but are not limited to, straight and branced alkyl and alkenyl groups containing functional groups such as carboxyl, amino, hydroxyl, and thiol, through which covalent bonds can be formed to connect the linker to the polymer and to other components. A preferred linker is a short peptide chain (H-[NHCHR—CO]n—OH) where n is 1-20, or alternatively from 1-18, or alternatively from 1-16, or alternatively from 1-14, or alternatively from 1-12, or alternatively from 2-14, or alternatively from 2-12, or alternatively from 3-20, or alternatively from 4-18, or alternatively from 5-20, or alternatively from 5-18, and R is the same or different for each of the n amino acids, and is one of the 22 side groups known to be present in natural amino acids, wherein the linker is the same or different and is selected from the group of a short peptide chain (H-[NHCHR—CO]n—OH) where n is 1-20 and R is the same or different for each of the n amino acids, and is one of the 22 side groups known to be present in natural amino acids; a short alkyl chain (CH₂)ₙ where n=2-10, terminated by two amino groups or two carboxyl groups or one amino group and one carboxyl group; an oligoethyleneoxy chain (CH₂CH₂O)ₙ where n=2-100, terminated by two amino groups or two carboxyl groups or one amino group and one carboxyl group; a poly(lactic-co-glycolic acid) (PLGA), polylactic acid (PLA), a polyglycolic acid (PGA) chain of average molecular weight of 2 kDa to 70 kDa terminated by two amino groups or two carboxyl groups or one amino group and one carboxyl group; —C(O)NH(CH₂)₆NH—; —C(O)NH(CH₂)₆NHC(O)CH₂—; —C(O)NH(CH₂CH₂O)₂CH₂CH₂NH—; —C(O)NH(CH₂CH₂O)₂CH₂CH₂NHC(O)CH₂[OCH₂CH₂]₂₃NH— or any combination thereof. A peptide linker can be incorporated into the polymer compound by one of the peptide condensation reactions (producing an amide bond) that are known in the art.

As used herein, “therapeutic agent” refers to a compound, mixture of compounds, or biologic agent that can provide a beneficial effect when administered to a patient.

As used herein, “amino acid” refers to a compound containing both amino (—NH₂) and carboxyl (—COOH) groups generally separated by one carbon atom. The central carbon atom may contain a substituent which can be either charged, ionisable, hydrophilic or hydrophobic. Any of 22 basic building blocks of proteins having the formula NH₂—CHR—COOH, where R is different for each specific amino acid, and the stereochemistry is in the ‘L’ configuration. Additionally, the term “amino acid” can optionally include those with an unnatural ‘D’ stereochemistry and modified forms of the ‘D’ and ‘L’ amino acids.

As used herein, “peptide” refers to a chain of amino acids in which each amino acid is connected to the next by a formation of an amide bond. Peptides are generally considered to consist of up to 30 amino acids, or alternatively up to 25 amino acids, or alternatively up to 20 amino acids, or alternatively up to 15 amino acids, or alternatively up to 10 amino acids, or alternatively up to 5 amino acids, or alternatively between about 5-10 amino acids, or alternatively between about 10-15 amino acids, while the term “protein” is applied to compounds containing longer amino acid chains.

As used herein, “glycoprotein” refers to a protein which contains a number of carbohydrate substituents.

As used herein, “halo” or “halogen” refers to fluorine (F), chlorine (Cl), bromine (Br) and iodine (I).

As used herein, a primary, secondary or tertiary alkyl amine refers to an RNH₂, an RR″NH or an RR′R″N group, wherein R, R′ and R″ independently represent, without limitation, alkyl, cycloalkyl, aryl, heteroaryl and heteroalicyclic moieties.

As used herein, “vitamin B12” or VB12″ (includes unless otherwise specified, VB12 derivatives and analogs of VB12) refers to the series of compounds otherwise know as cobalamins which are structurally identical and vary only in the nature of the monodentate axial ligand attached to the VB12 cobalt atom, which typically can be cyanide (cyanocobalamin), methyl (methylcobalamin), hydroxyl (hydroxycobalamin), or nitric oxide (nitrosylcobalamin). It is known in the art VB12 derivates can be made, for example by exchanging axial ligands under appropriate conditions, and such ligand exchange is incorporated as part of this disclosure. Non-limiting examples of VB12 derivatives include VB12-5′-O-carboxytriazole, VB12-5′-O-carboxylmidazole, VB12-5′-O-carboxyamido-C2-C20-alkylamines, VB12-5′-O-carboxyamido-oligoethyleneoxyamines, and dicarboxylic acid derivatives of the aforementioned compounds. Linkage of the VB12 to the lipids, nanoparticles and polymer systems to create the delivery systems described herein can be accomplished by converting one or more amide to carboxyl then using the free carboxyl to form a covalent link. Alternatively, formation of a covalent bond to one of the two hydroxyl groups of the ribose unit of VB12 can be employed. Alternatively, VB12 could be linked to the polymer system might also be accomplished by addition of a suitable monodentate ligand to the polymer, via an optional linker, and formation of a metal coordinate bond between the cobalt atom of VB12 and the polymer-attached monodentate ligand.

As used herein, a “disease” or “medical condition” is an abnormal condition of an organism that impairs bodily functions, associated with specific symptoms and signs.

As used herein, the term “cancer” refers to various types of malignant neoplasms, most of which can invade surrounding tissues, and may metastasize to different sites, as defined by Stedman's Medical Dictionary, 25th edition (Hensyl ed. 1990). Examples, without limitation, of cancers which may be treated using the compounds of the present invention include, but are not limited to, brain, ovarian, colon, prostate, kidney, bladder, breast, lung, oral, skin and blood cancers.

As used herein, a “tumor-seeking” group refers to an entity that is know to preferentially seek out and bind to surface structures on neoplastic cells that do not occur or are expressed to a substantially lesser degree by normal cells or entitles that preferentially accumulate in tumors over normal tissue.

As used herein, the terms “treat”, “treating” and “treatment” refer to a method of alleviating or abrogating a disease and/or its attendant symptoms. The effect may be prophylactic in terms of completely or partially preventing a disorder or sign or symptom thereof, and/or may be therapeutic in terms of a partial or complete cure for a disorder and/or adverse effect attributable to the disorder. For example, the life expectancy of an individual affected with a cancer will be increased and/or that one or more of the symptoms of the disease will be reduced.

As used herein, “administer,” “administering” or “administration” refers to the delivery of a compound or compounds of this invention or of a pharmaceutical composition containing a compound or compounds of this invention to a patient in a manner suitable for the treatment of a particular disease, such as cancer. “Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

A “patient” or a “subject” refers to any higher organism that is susceptible to disease. Examples of such higher organisms include, without limitation, mice, rats, rabbits, dogs, cats, horses, cows, pigs, sheep, fish and reptiles. In some embodiments, “patient” or “subject” refers to a human being.

As used herein, the term “therapeutically effective amount” refers to that amount of a compound or combination of compounds of this invention which has the effect of (a) preventing a disorder from occurring in a subject that may be predisposed to a disorder, but may have not yet been diagnosed as having it; (b) inhibiting a disorder, i.e., arresting its development; or (c) relieving or ameliorating the disorder. For example, but not limited to, (1) reducing the size of the tumor; (2) inhibiting (that is, slowing to some extent, preferably stopping) tumor metastasis; (3) inhibiting to some extent (that is slowing to some extent, preferably stopping) tumor growth; (4) relieving to some extent (or preferably eliminating) one or more symptoms associated with the cancer; and/or (5) extending survival time of the patient.

As used herein, a “pharmaceutical composition” refers to a mixture of one or more of the compounds of this invention with other chemical components such as pharmaceutically acceptable excipients or carrier. The purpose of a pharmacological composition is to facilitate administration of a compound of this invention to a patient.

As used herein, a “pharmaceutically acceptable excipient” or “pharmaceutically acceptable carrier” refers to an excipient that does not cause significant irritation to an organism and does not abrogate the biological activity and properties of the administered composition. “Pharmaceutically acceptable carrier” or “pharmaceutically acceptable excipient” refers to any diluents, excipients, or carriers that may be used in the compositions of the invention. Such excipients or carriers include, without limitation, ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

### MODES FOR CARRYING OUT THE INVENTION

In one aspect, the present invention relates to nanoparticles formed by bringing together, in a suitable solvent or solvent mixture under conditions which result in nanoparticle formation, one or more therapeutic agents with one or more synthetic, semi-synthetic or natural polymers in which vitamin B12 or a derivative thereof is attached via suitable linker groups to at least one polymer. Other physically-bound or covalently-linked molecules for targeting or delivery are optionally attached. Other components may be used which either assist in nanoparticle formation or in the placement of vitamin B12 or a derivative thereof on the surface of the nanocarrier. The polymers and the therapeutically active agents can form nanoparticles, either alone or in combination with the other aforementioned components.

In a further aspect, the present invention relates to a nanocarrier formed by bringing together, in a suitable solvent or solvent mixture under conditions which result in the nanocarrier formation, one or more synthetic, semi-synthetic or natural polymer in which vitamin B12 or a derivative thereof is attached via suitable linker groups to at least one polymer. Other physically-bound or covalently-linked molecules for targeting or delivery are optionally attached. Other components which either assist in nanoparticle formation or in the placement of vitamin B12 on the surface of the nanocarrier are optionally included. The polymers can form nanoparticles, either alone or in combination with the other aforementioned components. One or more therapeutic agents are infused into the nanocarriers following the formation of the nanocarrier to complete the drug delivery system.

In a further aspect, the present invention relates to nanoparticles formed by bringing together in a suitable solvent or solvent mixture crystalline or non-crystalline nanoparticles of the therapeutic agent or mixture of therapeutic agents with one or more synthetic, semi-synthetic or natural polymers in which vitamin B12 or a derivative thereof is attached via suitable linker groups to at least one polymer. Other physically-bound or covalently-linked molecules for targeting or delivery are optionally attached. Optionally, nanoparticle formation can utilize other components which either assist in coating of the nanoparticle with the polymer or in the placement of vitamin B12 on the surface of the nanoparticle. The polymers and the therapeutically active agents can form nanoparticles, either alone or in combination with the other aforementioned components.

In a further aspect, the present invention relates to nanoparticles formed by first forming a polymer nanoparticle of a therapeutic agent by bringing together in a suitable solvent or solvent mixture a solution of the therapeutic agent and one or more synthetic, semi-synthetic or natural polymers under conditions which result in nanoparticle formation. The resultant nanoparticles are subsequently coated with one or more synthetic, semi-synthetic or natural polymers in which vitamin B12 or a derivative thereof is attached via suitable linker groups to at least one polymer. Other physically-bound or covalently-linked molecules for targeting or delivery are optionally attached. Other components can be included which either assist in nanoparticle formation or in the placement of vitamin B12 on the surface of the nanocarrier. The polymers and the therapeutically active agents can form nanoparticles, either alone or in combination with the other aforementioned components.

In a further aspect, the present invention relates to nanoparticles formed by first forming a polymer nanoparticle of a therapeutic agent by bringing together in a suitable solvent or solvent mixture a solution of the therapeutic agent and one or more synthetic, semi-synthetic or natural polymers under conditions which result in nanoparticle formation. The resultant nanoparticles are optionally subsequently coated with one or more synthetic, semi-synthetic or natural polymers which may or may not contain covalently-linked molecules for targeting or delivery. Vitamin B12 or a derivative thereof is then attached via suitable linker groups by either physical or covalent binding to the surface of the nanoparticle. Other components can be included which either assist in nanoparticle formation or in the placement of vitamin B12 on the surface of the nanocarrier. The polymers and the therapeutically active agents can form nanoparticles, either alone or in combination with the other aforementioned components.

In a further aspect, the present invention relates to drug-loaded liposomes or micelles which are formed by procedures known in the art comprising a mixture of lipids (hydrophobic molecules with hydrophilic end groups) provided that some of the lipids forming the nanocarrier have vitamin B12 linked to the hydrophobic portion of the lipid via suitable linker groups. Other physically-bound or covalently-linked molecules for targeting or delivery are optionally attached. Other components can be included which either assist in liposome or micelle formation or in the placement of vitamin B12 or a derivative thereof on the surface of the nanocarrier. The lipids and the therapeutically active agents can form nanoparticles, either alone or in combination with the other aforementioned components.

In a further aspect, the present invention relates to drug-loaded liposomes, micelles, or nanoparticles are described above except that the drug-loaded liposomes, micelles, or nanoparticles are formed without vitamin B12 or a derivative thereof and/or without other physically-bound or covalently-linked molecules for targeting or delivery, and vitamin B12 or a derivative thereof and other optional physically-bound or covalently-linked molecules for targeting or delivery are covalently or physically attached after nanoparticle formation.

In a further aspect, the present invention relates to nanoparticles formed either from mixtures of polymers and drug or nanoparticles formed by coating of a drug or drug-polymer nanoparticle, as described above, whereby that polymers are cross-linked by use a suitable crosslinking agent or mixture of suitable cross-linking agents. Cross-linking agents can be introduced before, during, or after nanoparticle formation.

In a further aspect, the present invention relates to nanoparticles formed as described by one of the methods described above whereby the nanoparticle is formed in the absence of the drug and the drug is introduced into the preformed nanoparticle by diffusion.

In some embodiments, nanoparticles are formed by bringing together the components of the nanoparticle in an aqueous environment, although other solvent systems known in the art may also be used. The nanoparticles formed may be either soluble or insoluble in the solvent system. Nanoparticles can be isolated by techniques known in the art. For example, soluble nanoparticles can be isolated by precipitation with a cosolvent or by removal of solvent (e.g. evaporation, lyophilization or spray drying) optionally preceeded by a purification method such as tangential flow filtration (TFF) or centrifugal ultrafiltration. Insoluble nanoparticles might be isolated by centrifugation or filtration, also optionally preceded by a purification method such as TFF.

The solid nanoparticles formed and isolated as described above, may then be formulated for human or veterinary administration by standard methods. For example, optionally with suitable excipients, the nanoparticles might be formulated into tablets or capsules for oral administration, as lyophilized or dried formulations in vials for subsequent reconstitution with an injection vehicle and administration to humans or animals by injection, or as solutions or suspensions for administration to humans or animals by injection.

The pharmaceutical formulations of the nanoparticles of this invention can be used for oral drug delivery and/or disease targeted delivery of a wide variety of therapeutic agents, including, but not limited to, small and large synthetic molecules, proteins, peptides, glycoproteins, humanized and non-humanized monoclonal antibodies and therapeutically relevant fragments thereof, and agents for effecting the delivery of polynucleotides alone or in combination with a gene delivery vector. The polynucleotides, include for example those which are, or that encode RNA interference (RNAi) such as siRNA, miRNA dsRNA, mRNA and antisense RNA, as well DNA, such as in gene therapy applications.

The pharmaceutical formulations of the nanoparticles of this invention can be used to treat a wide variety of diseases including, but not limited to cancer, autoimmune conditions, endocrine disorders, diabetes, genetic conditions, chromosome conditions, viral infections, bacterial infections, parasitic infections, mitochondrial diseases, sexually transmitted diseases, immune disorders, balance disorders, pain, systemic disorders, blood conditions, blood vessel conditions, nerve conditions, and conditions of muscles, heart and other organs.

In one aspect, there is provided a nanoparticle comprising, or alternatively consisting essentially of a drug nanoparticle coated with one or more stable or degradable synthetic, semi-synthetic or natural polymers comprising one hydrophilic or hydrophobic substituents, and a vitamin B12 or a derivative thereof covalently linked to the nanoparticle via an optional linker group.

In one aspect, there is provided a nanoparticle comprising, or alternatively consisting essentially of a drug nanoparticle coated with one or more stable or degradable hydrophobic or hydrophilic synthetic, semi-synthetic or natural polymers, and a vitamin B12 or a derivative thereof covalently linked to the nanoparticle via an optional linker group.

In some embodiments, the synthetic, semi-synthetic or natural polymers have charged or ionizable functional groups, and such charged or ionizable groups can be the same or different.

In some embodiments, the nanoparticle of the above noted aspects further comprises one or more of components selected from the group consisting of polyethylene glycol (PEG), PEG block copolymers, polyacrylic, polymethacrylic, polyacrylamide, polymethacrylamide, synthetic polymer, polysaccharide, surfactant, and metal ions.

In some embodiments, the vitamin B12 or a derivative thereof is attached to one or more of the components.

In some embodiments, an average nanoparticle diameter is in a range of about 20 nm to about 800 nm.

In some embodiments, the nanoparticle is configured for oral administration in a subject.

In some embodiments, the nanoparticle is configured for administration by injection to a subject.

In some embodiments, the nanoparticle is configured for administration by intravenous injection or infusion to a subject.

In some embodiments, the nanoparticle is configured for administration by intraperitoneal injection or infusion to a subject.

In some embodiments, the nanoparticle is configured for subcutaneous administration to a subject.

In some embodiments, the nanoparticle is configured for administration by topical application to a subject.

In some embodiments, the nanoparticle is configured for administration by topical application to a mucosal surface of a subject.

In some embodiments, the nanoparticle is configured for administration by topical application to the skin of a subject.

In some embodiments, the nanoparticle is configured for administration by application to the surface of the eye of a subject.

In some embodiments, one or more of the polymers can be a linear, brached or cross-linked polysaccharide such as dextran, cellulose, starch, chitosan, chondrotin, glycosaminoglycan, and derivatives thereof.

In some embodiments, one or more of the polymers is a polyester, a polyanhydride, a peptide, or a protein

In some embodiments, one or more of the polymers is a biologically-derived protein or glycoprotein such as bovine or human albumin.

In some embodiments one or more of the polymers is polylactic acid (PLA), polyglycolic acid (PGA), or polylactic glycolic acid (PLGA)

In some embodiments, the VB12 is a VB12 derivative wherein an axial ligand substituent on a cobalt atom of vitamin B12 is CN, Me, OH or NO.

In some embodiments, the therapeutic agent is selected from the group consisting of a small or large synthetic or semi-synthetic molecule, protein, peptide, glycoprotein, nucleoside, nucleotide, humanized monoclonal antibody, non-humanized monoclonal antibody, therapeutically relevant fragments of humanized and/or non-humanized monoclonal antibody, and agents for effecting RNA interference (RNAi) such as dsRNA, miRNA, siRNA and antisense RNA.

A siRNA can be designed following procedures known in the art. See, e.g., Dykxhoorn, D. M. and Lieberman, J. (2006) “Running Interference: Prospects and Obstacles to Using Small Interfering RNAs as Small Molecule Drugs,” Annu Rev. Biomed. Eng. 8:377-402; Dykxhoorn, D. M. et al. (2006) “The silent treatment: siRNAs as small molecule drugs,” Gene Therapy, 13:541-52; Aagaard, L. and Rossi, J. J. (2007) “RNAi therapeutics: Principles, prospects and challenges,” Adv. Drug Delivery Rev. 59:75-86; de Fougerolles, A. et al. (2007) “Interfering with disease: a progress report on siRNA-based therapeutics,” Nature Reviews Drug Discovery 6:443-53; Krueger, U. et al. (2007) “Insights into effective RNAi gained from large-scale siRNA validation screening,” Oligonucleotides 17:237-250; U.S. Patent Application Publication No.: 2008/0188430; and U.S. Patent Application Publication No.: 2008/0249055.

siRNAs can be made with methods known in the art. See, e.g., Dykxhoorn, D. M. and Lieberman, J. (2006) “Running Interference: Prospects and Obstacles to Using Small Interfering RNAs as Small Molecule Drugs,” Annu Rev. Biomed. Eng. 8:377-402; Dykxhoorn, D. M. et al. (2006) “The silent treatment: siRNAs as small molecule drugs,” Gene Therapy, 13:541-52; Aagaard, L. and Rossi, J. J. (2007) “RNAi therapeutics: Principles, prospects and challenges,” Adv. Drug Delivery Rev. 59:75-86; de Fougerolles, A. et al. (2007) “Interfering with disease: a progress report on siRNA-based therapeutics,” Nature Reviews Drug Discovery 6:443-53; Krueger, U. et al. (2007) “Insights into effective RNAi gained from large-scale siRNA validation screening,” Oligonucleotides 17:237-250; U.S. Patent Application Publication No.: 2008/0188430; and U.S. Patent Application Publication No.: 2008/0249055.

A siRNA may be chemically modified to increase its stability and safety. See, e.g. Dykxhoorn, D. M. and Lieberman, J. (2006) “Running Interference: Prospects and Obstacles to Using Small Interfering RNAs as Small Molecule Drugs,” Annu Rev. Biomed. Eng. 8:377-402 and U.S. Patent Application Publication No.: 2008/0249055.

In some embodiments, the therapeutic agent is selected from the group consisting of analgesic, antiallergenic, antianginal agent, antiarrythmic drug, antibiotic, anticoagulant, antidementia drug, antidepressant, antidiabetic, antihistamine, antihypertensive, anti-inflammatory, antineoplastic agent, antiparasitic, antipyretic, antiretroviral drug, antiulcerative agent, antiviral agent, cardiovascular drug, cholesterol-lowering agent, CNS active drug, a hormone, growth hormone inhibitor, growth hormone, hematopoietic drug, hemostatic, hypotensive diuretic, keratolytic, therapeutic for osteoporosis, vaccine, vasoconstrictor, and vasodilator.

In one aspect, there is provided a process for preparing a nanoparticle composition comprising the nanoparticle of any of the above recited aspects and embodiments, comprising, or alternatively consisting essentially of or alternatively consisting of combining the one or more synthetic or natural polymers, the therapeutic agent, and the vitamin B12 or a derivative thereof, in a suitable solvent, and isolating, purifying and/or drying the nanoparticles. In some embodiments, the solvent is >50% water.

In another aspect, there is provided a process for preparing a nanoparticle composition comprising the nanoparticle of any of the above recited aspects and embodiments, comprising, or alternatively consisting essentially of or alternatively consisting of mixing two immiscible solvents and a surfactant to produce an emulsion, optionally cross-linking the nanoparticles, and isolating, purifying, and/or drying resultant nanoparticles.

In some embodiments, the nanoparticles are isolated by solvent evaporation, spray-drying or lyophilization.

In some embodiments, the nanoparticles are isolated by filtration or centrifugation

In some embodiments, the nanoparticles are isolated by addition of a cosolvent followed by filtration or centrifugation.

In some embodiments, the purifying step is effected by washing the nanoparticles with a suitable solvent.

In some embodiments, the above recited aspects further comprise modifying the nanoparticles to effect cross-linking of the components of the nanoparticle.

In some embodiments, the above recited aspects further comprise modifying the nanoparticles to add a vitamin B12 analog or a derivative thereof to a surface of the nanoparticle by physical or covalent attachment.

In some embodiments, the above recited aspects further comprise modifying the nanoparticles to substitute an axial ligand on a one or more cobalt atoms of attached vitamin B12 with replacement axial ligands.

In another aspect, there is provided a pharmaceutical composition comprising the nanoparticle of the above recited aspects, and a pharmaceutically-acceptable excipient or carrier.

In some embodiments, the composition is formulated as a tablet, a capsule, or a liquid.

In some embodiments, the composition is formulated as a lyophilized powder in a container for subsequent re-suspension or dissolution of the pharmaceutical composition in a pharmaceutically-acceptable injection vehicle.

In some embodiments, the composition is formulated as a suspension or solution in a pharmaceutically-acceptable injection vehicle.

In another aspect, there is provided a method for treating a subject, comprising, or alternatively consisting essentially of, or alternatively consisting of, administering an effective amount of the nanoparticle of any of the above recited aspects or the pharmaceutical composition of any of the above recited aspects.

In some embodiments, the therapeutic agent is an anti-diabetic agent.

In some embodiments, the therapeutic agent is a hormone.

In some embodiments, the therapeutic agent is an anti-neoplastic agent.

In some embodiments, the nanoparticles of this invention are made by a solvent extraction/evaporation method or modification of that method.

In some embodiments, the nanoparticles of this invention are made by coating of crystalline or non-crystalline particles with polymers described herein by the extraction/evaporation method or modification of that method such that the polymer or polymer mixture creates a shell around the active pharmaceutical agent and the polymer-coated particle remains in nanoparticle size range.

### Oral Drug Delivery

A number of technologies have been advocated for the enhancement of oral bioavailability of pharmaceutically-active compounds. As an example, one area of particularly active research has been in the development of technologies for the oral delivery of insulin. Khafagy et al (Advanced Drug Delivery Reviews 59 (2007) 1521-1546) classified the various oral insulin approaches as: Absorption enhancers; Enzyme inhibitors; Mucoadhesive polymeric systems; Particulate carrier delivery systems; and Targeted delivery systems.

Absorption or permeation enhancers are molecules which either increase the fluidity of membranes or widen junctions between the cells of membranes thus providing a small transient improvement in paracellular and transcellular drug transport. There are a number of distinct disadvantages to absorption enhancers for oral drug delivery:

Typically, they should slightly precede the appearance of drug molecules at the absorption site to provide maximum possible drug absorption. Once the concentration of the enhancer molecule decreases at the membrane site (for example, by continued transit in the GI tract, or by virtue of the fact that it is itself absorbed or metabolized), the membrane permability returns to normal.

Increasing membrane permeability permits increased penetration of all molecules in the vicinity, not just the drug molecules.

Enzyme inhibitors slow the rate at which actives, particularly proteins and peptides, are enzymatically degraded in the GI tract. In principle, this provides for a higher concentration of the active at the sites of absorption, resulting in greater passive absorption by virtue of a larger concentration gradient. This effect is only beneficial for actives that are naturally able to diffuse readily across the gut wall, and are only prevented from doing so through enzymatic degradation of the active compound. Additionally, inhibition of enzyme activity in the GI tract can give rise to significant adverse effects as inhibition of protein degradation will be non-selective. For example, enzyme inhibitors will reduce the rate to breakdown (and hence reduced absorption) of food proteins.

Peristalsis generates a flow of material down the GI tract. Materials moving along the small intestine, where most pharmaceutical actives are thought to be absorbed, do so in an average time of about three hours. If were possible to retard the flow of drugs, and provide them with greater contact at the sites of absorption, it should be possible to achieve higher levels of absorption of drugs that are otherwise poorly absorbed in the GI tract. Because of transient ‘sticking’ of mucoadhesive polymeric systems to the mucosal surface of the GI tract lumen, formulations based upon such polymers have the potential to demonstrate an extended residence on the epithelial cell layer, slowing the flow of these particles relative to other material in the GI tract. When formulated into particles, mucoadhesive polymers may also provide some protection to embedded active agents that might otherwise be degraded in the GI tract. Because of the direct contact between the polymer formulation and the GI mucosa, other potential advantages of this oral drug delivery system is the possibility for direct diffusion of actives from the particle into the mucosa and epithelial cell layer, and for pinocytosis of particles into epithelial cells. All of these potential benefits suggest that oral drug delivery systems based upon mucoadhesive polymers should be highly effective, yet results to date in numerous examples in the literature indicate only modest improvements in oral bioavailability of pharmaceutical active compounds using mucoadhesive polymer formulations.

Gastrointestinal absorption of many essential nutrients and vitamins can be facilitated by active transport processes. These processes generally require the material to bind to a surface receptor, which initiates a process such as receptor-mediated endocytosis whereby the active is absorbed into the epithelial cell. Disassociation of the receptor-active complex occurs and other processes then facilitate the transfer of the active material into the blood stream. One transport system which has been well documented in the literature is the process for absorption of vitamin B12 (VB12). VB12 liberated from food binds to intrinsic factor (IF, which is produced in the stomach and passes down the GI tract following a meal), and the VB12-IF complex binds to the Cubulin receptor, primarily located in the ileum. Receptor-mediated endocytosis, as described above, then takes place. Dissociation of the receptor-IF-VB12 complex in the epithelial cell results in liberation of VB12, which then binds to transcobalamin II, a protein which facilities the transfer of VB12 to the blood stream.

It has been documented by Russell-Jones and others that the VB12 uptake mechanism in the GI tract can be used to facilitate the oral absorption of other compounds. Using a ‘Trojan Horse’ approach, the active is either covalently linked via a degradable linker group to VB12, or covalently linked via a degradable linker group to a polymer which is also linked to VB12, or encapsulated in a nanoparticle to which VB12 is attached (see FIG. 2). In the polymer approach, multiple drug-linker groups can be attached to a single polymer strand. For each of these possibilities, provided that VB12 is bound to the linker or particle so as not to prevent binding to IF, these constructs will bind IF in the GI tract and be taken up primarily in the ileum by the cubulin receptor and transported to the bloodstream. Breakdown of the degradable linker will then release drug in the bloodstream, completing its oral absorption. Similarly, drug release by diffusion from the nanoparticle and/or breakdown of the nanoparticle structure in the bloodstream will result in bioavailability of the active. In the case of single conjugation of the active to the VB12 via a linker, one molecule of the drug is absorbed for each receptor-mediated endocytotic event. By comparison, the polymer approach allows for multiple drug molecules to be absorbed each time one polymer strand is absorbed as a result of VB12 attached to that polymer strand binding to IF and cubulin. This allows for an ‘amplification’ of oral uptake when compared with the 1:1 conjugate. Similarly, a VB12 nanoparticle can carry many copies of the drug, also permitting amplification of drug uptake.

A number of patents which describe either single VB12 conjugates, VB12-polymer conjugates, and VB12-coated naoparticles are known, represented by the following (all of which are incorporated herein by reference in their entirety): U.S. Pat. Nos. 5,428,023, 5,449,720, 5,548,064, 5,574,018, 5,589,463, 5,807,832, 5,863,900, 5,869,466, 5,589,463, 6,083,926, 6,150,341, 6,159,502, 6,221,397, 6,262,253, 6,482,413.

In some embodiments, the formation of a covalent link to connect the drug to VB12 may not be the preferred method of utilizing this technology. By formation of a covalent link to the drug, it may be chemically altered. For a drug which has already received Regulatory approval for its use as a medication, a new active pharmaceutical ingredient (API) would have been created that will require a full drug development program for its approval. The release of the drug requires cleavage of the degradable linker, which may leave fragments of the linker still attached to the drug, such that is a different chemical entity. By trapping the drug in a VB12-coated nanoparticle, it remains chemically unaltered, so a previously approved drug should not need a full development program for Regulatory approval of the VB12-coated nanoparticle formulation of that drug.

Many methods of forming nanoparticles and utilizing such nanoparticles for drug delivery are reported in the literature. Furthermore, VB12-coated nanoparticles for oral drug delivery have been described (U.S. Pat. Nos. 6,159,502; 6,482,413; and publication No. WO2007131286). None of the formulations described in these patents have advanced from basic research to the clinic as each of these technologies has fundamental technical issues; as examples poor encapsulation/weak binding of the drug to the carrier. The methods of preparation known in the art can also give rise to degradation or denaturing of protein, peptides and other pharmaceutical active ingredients, lowering efficacy and introducing additional impurities.

It is an object of the present invention to overcome or at least alleviate one or more of the above-mentioned disadvantages of the prior art.

### Disease-Targeting

In many diseases which involve cell proliferation, there is increased demand for certain vitamins compared with normal tissue. This phenomenon can be utilized for targeting drugs to the site of disease such as tumors. For example, folic acid (vitamin B9), riboflavin, thiamine, and vitamin B12 have been reported and used to target drugs and radioactive materials to tumors for therapy and diagnosis (U.S. Pat. Nos. 5,108,921, 5,416,016, 5,635,382, 5,688,488, 7,128,893, 7,601,332, and Waibel et al, Cancer Res., 2008, 68, 2904-2911). In most cases, the drug is covalently linked to the targeting system, thereby altering the drug and potentially altering its pharmacological and toxicological profile. A simple method is required to target the drug to sites of disease without chemical modification of the drug.

In many diseases, cells have an increased demand for vitamin B12 which is reflected by an increase in the expression of cell surface receptors which facilitate the uptake, through receptor-mediated endocytosis, of this vitamin. Mechanistically, vitamin B12 binds to the circulating protein, transcobalamin II (TC-II), and it is the B12-TC-II complex which is recognized by the cell surface receptors. The B12-TC-II complex binding results in receptor-mediated endocytosis and internalization of the complex, followed by release of the vitamin B12. As was the case for vitamin B12 uptake in the GI tract, the process for cell uptake of vitamin B12 can be utilized using the ‘Trojan Horse’ principle to transport molecules into cells when these molecules are chemically linked to vitamin B12. For example, R. Waibel et al, Cancer Res., 2008, 68, 2904-2911.

It is one object of the present invention to provide drug carrier systems and formulations which are effective while requiring no drug modification.

### Polyelectrolyte Complexes

The polymers for use in this invention are, in one aspect, capable of forming a polyelectrolyte complex. Polyelectrolyte complex (PEC) is a term which relates to two or more compounds binding to each other by virtue of multiple charge interactions. For the formation of nanoparticle PECs, it is usual that at least one of the compounds involved in an oligomer or polymer that contains multiple charged (or ionisable) groups, all either positive or negative. This polymer, when brought into contact with an compound containing one or more charged (or ionisable) groups of the opposite charge forms a complex wherein the charged groups on one compound form ionic bonds with the charged groups of the other compound. Typically, both compounds possess charged or ionisable groups and form multiple ionic bonds with each other. Further interactions such as hydrophobic bonding and H-bonding may serve to increase the strength of binding of one compound to the other. In the formation of nanoparticles, many molecules of the two or more charged or ionisable compounds come together to form a three-dimensional matrix of nanoparticle size. In the case of drug-loaded PECs, a simple example might be a pharmacologically-active peptide with either a net positive or net negative overall charge at a suitable pH with a polymer which has charged (or ionisable) groups which have the opposite charge to that of the peptide. Another example of PECs results from the formation of PECs from two polymers, one with negatively charged groups and one with positively-charged groups. Bringing these two polymers into contact in an aqueous environment which also contains the drug can result in the formation of PECs in which the drug is trapped in the nanoparticle matrix during PEC formation. In either of these two general examples, controlled drug release can result through slow disassociation of the nanoparticle in the body. The PEC components can be chemically-modified to assist in nanoparticle formation; for example, conversion of a tertiary to quaternary amine or through the addition of hydrophobic groups

Therapeutic agents ideally suited for formation of drug-loaded PECs are either highly charged (such as oligonucleotides) or contain multiple charged groups that can form ionic bonds with the charged carrier polymers. Additionally, therapeutic agents need to be stable under the conditions of formation and storage of PECs. PECs are typically manufactured in an aqueous environment and have water molecules contained in the nanoparticle matrix. Such conditions are less than ideal for many therapeutic agents, and so it is desirable to have other nanocarrier systems other than PECs which are better suited for the stability and in vivo controlled release of therapeutic agents for which PECs are unsuited.

A patent application has been filed which describe VB12 PECs as drug delivery vehicles (U.S. Ser. No. 61/378,272, Multivitamin Targeting of RNAi Therapeutics) which is incorporated herein by reference in its entirety.

### Nanocarriers

It is one object of the present invention to provide novel nanocarrier systems and simple methods of preparation whereupon a nanoparticle is formed presenting molecules of vitamin B12 or a derivative thereof on the surface of said nanoparticle and one or more therapeutically-active compounds are contained within the nanoparticle.

It is an additional object of the present invention that the above nanocarrier system provides some protection from degradation or denaturing of the one or more therapeutically-active compounds contained within the nanoparticle in body compartments in body compartments in which one or more therapeutically-active compounds contained within the nanoparticle might otherwise, if unprotected, be caused to degrade, denature or metabolize.

It is an additional object of the present invention that the above nanocarrier system has the potential benefit of transportation from one body compartment to another by utilizing the body's natural transportation mechanisms for vitamin B12, including, but not limited to, transportation from the gut lumen to the portal blood vein in the ileum of the GI tract, passage across cell membranes to enter cellular compartments, and traverse major biological barriers such as the blood-brain barrier.

It is an additional object of the present invention that the above nanocarrier system can release the one or more therapeutically active compounds contained within the nanoparticle in a controlled manner, and that compound release can result from diffusion of drug through the nanoparticle matrix and/or degradation of the matrix.

It is an additional object of the present invention that the above nanocarrier system can release the one or more therapeutically active compounds contained within the nanoparticle at sites within the body to achieve a therapeutically-meaningful effect.

It is an additional object of the present invention that the above nanocarrier system can degrade in the body to permit the components of the nanoparticle to be safely metabolized and eliminated from the body.

It is an additional object of the present invention that the above nanocarrier system can formulated by methods known in the art to provide pharmaceutical preparations suitable for administration to patients. Examples of pharmaceutical preparations that might be suitable for the nanocarrier system of this invention include, but are not limited to, tablets or capsules for oral administration, lyophilized powers in vials for subsequent reconstitution with a pharmaceutically-acceptable vehicle for injection into the patient, or liquids comprising the drug-containing nanocarrier system in pharmaceutically-acceptable vehicle for injection into the patient.

It is an additional object of the present invention that the above nanocarrier system be administered to patients for the prevention and treatment of diseases, including, but not limited to cancer, autoimmune conditions, endocrine disorders, diabetes, genetic conditions, chromosome conditions, viral infections, bacterial infections, parasitic infections, mitochondrial diseases, sexually transmitted diseases, immune disorders, balance disorders, pain, systemic disorders, blood conditions, blood vessel conditions, nerve conditions, and conditions of muscles, heart and other organs.

In one mode, the present invention consists of nanoparticles formed by bringing together in a suitable solvent one or more synthetic, semi-synthetic or natural polymers with a therapeutic agent. One or more of the polymers will contain vitamin B12 (VB12) or a derivative thereof covalently bound to the polymer via a suitable linker. Optionally, one or more polymers will contain other physically-bound or covalently-linked molecules for targeting or delivery. Formation of nanoparticles may optionally utilize other components which either assist in nanoparticle formation or in the placement of VB12 on the surface of the nanoparticle. The polymers and the therapeutically active agent can form a nanoparticle, either alone or in combination with the other aforementioned components. VB12 is an essential component of the nanoparticle, introduced prior to nanoparticle formation either by covalent attachment to the polymer, to the therapeutic agent, and/or to one of the optional additional components.

In a further mode, the present invention consists of nanoparticle shells formed by coating a nanoparticle of the therapeutic agent with polymers. The nanoparticle of the therapeutic agent can comprise crystalline or non-crystalline form of the therapeutic agent or a mixture of the therapeutic agent with one or more polymers. The nanoparticle of the present invention is formed by coating the nanoparticle of the therapeutic agent in a suitable solvent with one or more synthetic, semi-synthetic or natural polymers. One or more of the coating polymers will contain VB12 or a derivative thereof covalently bound to the polymer via a suitable linker. Optionally, one or more coating polymers will contain other physically-bound or covalently-linked molecules for targeting or delivery. Formation of nanoparticles of the present invention may optionally utilize other components which either assist in nanoparticle formation or in the placement of VB12 on the surface of the nanoparticle.

In a further mode, the present invention consists of micelles or liposomes formed by lipids encapsulating the therapeutic agent. The micelle or liposome is formed from components and by methods known in the art in which some of the lipids will contain VB12 covalently bound to the lipid via a suitable linker. Optionally, one or more lipids will contain other physically-bound or covalently-linked molecules for targeting or delivery. Formation of micelles or liposomes of the present invention may optionally utilize other components which either assist in nanoparticle formation or in the placement of VB12 on the surface of the nanoparticle.

In a further mode, the present invention consists of nanocarriers, nanoparticle shells, micelles or liposomes formed as described above in which VB12 is not a component or part of a component of the nanocarrier, nanoparticle shell, micelle or liposome and is introduced to the surface of the nanocarrier, nanoparticle shell, micelle or liposome after its formation either by formation of a covalent bond between the surface of the nanocarrier, nanoparticle shell, micelle or liposome and VB12 or VB12 derivative, or by the formation of a physical bonds (ionic, hydrophilic, and/or hydrophobic) between the nanocarrier, nanoparticle shell, micelle or liposome and VB12 or VB12 derivative.

As described earlier, vitamin B12 contains a monodentate axial ligand. It is known in the art that these axial ligands can be exchanged under appropriate conditions, and such ligand exchange is incorporated as part this disclosure. For example, it is known that nitrosyl cobalamin can be effective as an antitumor agent because it serves to deliver nitric oxide to tumors (for example; Bauer, Anti-Cancer Drugs, 1998, 9, 239) and it may be desirable to convert VB12 in the nanoparticles of this invention to the nitrosyl form to enhance the therapeutic effect. In addition, in order to link the VB12 molecule to a polymer via an optional linker, the VB12 may be connected to the linker through the cobalt atom of VB12 by way of a ligand exchange process, as described in (for example; U.S. Application 20020115595; Bagnato et al, J. Org. Chem. 2004, 69, 8987).

Alternatively VB12 can be attached using other methods known in the art. For example, one or more of the primary amide groups of VB12 may be selectively hydrolyzed to generate a free carboxyl group or ester, and subsequently the VB12 can be linked to the polymer via an optional linker through the liberated carboxyl group by methods well-known in the art (for example; Wilbur et al, Bioconjugate Chem. 1996, 7, 461-474). The preferred method of attachment of VB12 to the polymer via an optional linker involves the formation of a covalent bond to one of the two hydroxyl groups of the ribose unit of VB12 by methods known in the art (for example; McEwan et al, Bioconjugate Chem. 1999, 10, 1131-1136).

Examples of polymers that can be used to form the nanocarriers and nanoparticle shells of this invention include, but are not limited to, polylactic acid (PLA), polyglycolic acid (PGA), polylactic-glycolic acid (PLGA), polyvinylalcohol (PVA), polyanhydrides, polyacylates, polymethacrylates, polyacylamides, polymethacrylate, dextran, chitosan, cellulose, starch, dendrimers, peptides, proteins, polyethyleglycols, and synthetic derivatives of the aforementioned polymers as well as a polymer capable of forming a polyelectrolyte complex (PEC). For the purpose of fulfilling the requirements of this invention, the polymers may be optionally modified by covalent linkage of one or more VB12 molecules, either directly or via a suitable linker.

Examples of lipids that can be used to form the micelles and liposomes of this invention include, but are not limited to, straight or branched alkanes or alkene functionalized at one end by hydrophilic groups that may be charged or neutral. For the purpose of fulfilling the requirements of this invention, the lipids may be optionally modified by covalent linkage of one or more VB12 molecules, either directly or via a suitable linker. Suitable lipids include, but are not limited to, both single chain amphiphiles and double chain amphiphiles, such as phospholipids (e.g. phosphatidylcholine), Other components such as cholesterol, fatty acids and other lipid soluble molecules which are known in the art to modify the properties of liposomes and micelles can also be used in the formation of nanocapsules of this invention.

It is within the scope of this invention that naturally-occurring polymers or readily-available synthetic polymers be used directly for formation of nanocarriers of this invention, or that such polymers can be synthetically-modified. Modifications can include, but are not limited to, the introduction of charged or ionizable groups, attachment of VB12, and the introduction of functional groups (for example, hydrophobic or hydrophilic) which either enhance the nanocarrier formation and/or the pharmaceutical qualities of the resultant nanocarriers.

It is within the scope of this invention that naturally-occurring lipids or readily-available synthetic lipids be used directly for formation of nanocarriers of this invention, or that such lipids can be synthetically-modified. Modifications can include, but are not limited to, the introduction of charged or ionizable groups, attachment of VB12, and the introduction of functional groups (for example, hydrophobic or hydrophilic) which either enhance the nanocarrier formation and/or the pharmaceutical qualities of the resultant nanocarriers.

In some embodiments, a ratio of the therapeutic agent to the vitamin B12 in the nanocarriers of the present invention is in a range of 1:20 to about 20:1, or alternatively in a range of about 1:15 to about 15:1, or alternatively in a range of about 1:10 to about 10:1, or alternatively in a range of about 1:5 to about 5:1, or alternatively in a range of about 1:2 to about 2:1, or alternatively the ratio of the therapeutic agent to the vitamin B12 in the nanoparticles of the present invention is about 1:1, or alternatively about 2:1, or alternatively about 1:2, or alternatively about 3:1, or alternatively about 1:3, or alternatively about 4:1, or alternatively about 1:4, or alternatively about 5:1, or alternatively about 1:5, or alternatively about 6:1, or alternatively about 1:6, or alternatively about 7:1, or alternatively about 1:7, or alternatively about 8:1, or alternatively about 1:8, or alternatively about 9:1, or alternatively about 1:9, or alternatively about 2:3.

It will be obvious to those skilled in the art that pharmaceutically-suitable nanoparticles can also be formed by use of more than one polymer of a particular type. For example, in forming a nanocarrier, a synthetic polymer and a semi-synthetic polymer together to enable formation of a nanocarrier.

Furthermore, it will be obvious to those skilled in the art that pharmaceutically-suitable nanocarriers can also be formed by incorporation of more than one therapeutically-active compound.

As indicated above, it may be desirous in the formation of nanocarriers to utilize additional components before, during or after nanocarrier formation in order to control the size of nanoparticles, control stability and/or the drug release profile. Possible additional components include, but are not limited to, polyethylene glycol (PEG) and PEG block copolymers, polyacrylic, polymethacrylic, and other synthetic polymers, starch, cellulose, and other polysaccharides, fatty acids and other surfactants, and metal ions, especially di- and trivalent ions such as zinc, magnesium, and calcium. Additional components might also include a crosslinking agent, for example epoxy compounds, dialdehyde starch, glutaraldehyde, formaldehyde, dimethyl suberimidate, carbodiimides, succinimidyls, diisocyanates, acyl azide, reuterin, and crosslinking effected by ultraviolet irradiation.

As indicated above, it may be desirous in the formation of nanocarriers to utilize additional components before, during or after nanocarrier formation in order to improve the targeting or other biological properties of the nanoparticles. These components may be covalently or physically bound to polymers or other components of the nanocarrier and their purpose is to be present on the surface of the nanocarriers as well as VB12, in sufficient quantities to provide additional targeting options or favorably improve the pharmacokinetic or pharmacodynamic properties of the nanocarrier. Such additional components are known in the art and can include, but are not limited to, B vitamins other than VB12, proteins and peptides such as interferon, albumin, and monoclonal antibodies or their fragments thereof, peptides or other substances with which enable or assist in transmembrane transfer, mucoadhesive compounds, and compounds such as polyethylene glycol (PEG) and PEG block copolymers, which reduce nanoparticle uptake by the reticuloendothelial system (RES).

Also as indicated above, unless VB12 is bound to the nanoparticle after nanoparticle formation, then one of the components used in the formation of the nanoparticle must contain VB12 either covalently of physically linked to that component. VB12 might be linked, directly or via a suitable linker, to one or more of the component polymers, the therapeutically-active compound, or one of the additional components (if employed).

It is within the scope of this invention that the primary purpose of the additional component is to facilitate the introduction of VB12 to the nanoparticle during its formation. For example, the additional component could be VB12 which contains a fatty acid attached to either the 5′-O or 2′-O position (or both), and the VB12 is incorporated by hydrophobic interaction of the fatty acid portion with other hydrophobic components involved in nanoparticle formation. Other methods of incorporating VB12 as one of the additional components will be obvious to those skilled in the art. As another example, the VB12 additional component may be functionalized with a compound that is known to bind strongly to one of the other components of nanoparticle formation (e.g. strepatavidin and biotin are well known to bind strongly to each other; similarly, U.S. Pat. No. 5,605,890 exemplifies a cyclodextrin-adamantane “lock and key” binding system).

The polymers used in this invention can have an average molecular weight in the range of 1-10,000 kDa. The preferred average molecular weights will be determined by the specific requirements of formation and the desired pharmaceutical properties of the nanoparticles. In some embodiments, the average molecular weight of the polymer of the invention is in a range of about 1-10,000 kDa; or alternatively in a range of about 1-5,000 KDa; or alternatively in a range of about 1-1,000 KDa; or alternatively in a range of about 1-500 KDa; or alternatively in a range of about 1-100 KDa; or alternatively in a range of about 10-10,000 KDa; or alternatively in a range of about 10-5000 KDa; or alternatively in a range of about 10-4000 KDa; or alternatively in a range of about 10-2000 KDa; or alternatively in a range of about 10-1000 KDa; or alternatively in a range of about 10-500 KDa; or alternatively in a range of about 50-10,000 KDa; or alternatively in a range of about 50-5,000 KDa; or alternatively in a range of about 50-1,000 KDa; or alternatively in a range of about 50-500 KDa; or alternatively in a range of about 100-10,000 KDa; or alternatively in a range of about 100-5,000 KDa; or alternatively in a range of about 100-1,000 KDa; or alternatively in a range of about 100-500 KDa; or alternatively in a range of about 500-10,000 KDa; or alternatively in a range of about 500-1,000 KDa; or alternatively in a range of about 1000-10,000 KDa; or alternatively in a range of about 1000-5,000 KDa; or alternatively in a range of about 2000-10,000 KDa; or alternatively in a range of about 2,000-5,000 KDa; or alternatively in a range of about 4,000-10,000 KDa; or alternatively in a range of about 4000-5000 KDa; or alternatively in a range of about 5,000-10,000 KDa; or alternatively in a range of about 6,000-10,000 KDa; or alternatively in a range of about 7,000-10,000 KDa; or alternatively in a range of about 8,000-10,000 KDa; or alternatively in a range of about 9,000-10,000 KDa.

In one embodiment, a function of the nanoparticles of this invention is to facilitate or enhance the oral bioavailablity of the therapeutically active compound (or compounds) contained within the nanoparticle. For example, the therapeutically active compound (or compounds) may have poor natural oral bioavailability by virtue of either (or both) degradation or denaturing in the GI tract or an inability to cross the gut wall and enter the bloodstream.

In a further embodiment, a function of the nanoparticles of this invention is to modify the oral bioavailablity of the therapeutically active compound (or compounds) contained within the nanoparticle. For example, the therapeutically active compound (or compounds) may have sufficient oral bioavailability to be therapeutically effective when given orally, and the nanoparticles of this invention either improve oral bioavailability (reducing the amount of drug that needs to be administered) and/or alters the pharmacokinetic profile of the drug in a desirable manner.

In a further embodiment, a function of the nanoparticles of this invention is to facilitate targeting of the therapeutically active compound (or compounds) contained within the nanoparticle to sites of disease, especially in diseases in which the demand for VB12 is increased compared with the demand for the vitamin normally. Examples of diseases which are known to display increased demand for VB12 include cancer, rheumatoid arthritis, psoriasis, acute leukemia, lymphomas, Crohn's disease, ulcerative colitis, and multiple sclerosis. Pharmaceutical preparations for targeted delivery to sites of disease can be administered by injection.

In a further embodiment, a function of the nanoparticles of this invention is to combine oral drug delivery and targeting; following oral drug delivery as described above, the nanoparticles are then targeted to sites of disease, also as described above.

In a further embodiment, a function of the nanoparticles of this invention is to deliver polynucleotides (e.g. siRNA and antisense RNA) and other RNA interference therapeutics across cell membranes to deliver the actives into the intracellular environment and to the nucleus, where they are effective, and for gene therapy.

In a further embodiment, a function of the nanoparticles of this invention is to deliver therapeutics which are effective in the treatment of CNS disorders across the blood-brain barrier.

Therapeutic agents that can be delivered in effective amounts across biological barriers using the nanoparticles of this invention include, but are not limited to small molecules, macromolecules, synthetic drugs, semi-synthetic drugs, naturally-occurring compounds, proteins, peptides, nucleosides, nucleotides, analgesics, antiallergenics, antianginal agents, antiarrythmic drugs, antibiotics, anticoagulants, antidementia drugs, antidepressants, antidiabetics, antihistamines, antihypertensives, anti-inflammatories, antineoplastic agents, antiparasitics, antipyretic, antiretroviral drugs, antiulcerative agents, antiviral agents, cardiovascular drugs, cholesterol-lowering agents, CNS active drugs, growth hormone inhibitors, growth hormones, hematopoietic drugs, hemostatics, hormones, hypotensive diuretics, keratolytics, therapeutics for osteoporosis, vaccines, vasoconstrictors, vasodilators. Such therapeutics can be used alone or in combination with other therapeutic agents using dosing regimens effective in providing a beneficial therapeutic effect.

Examples of therapeutic agents that are analgesics are morphine, hydromorphone, oxymorphone, lovorphanol, levallorphan, codeine, nalmefene, nalorphine, nalozone, naltrexone, buprenorphine, butorphanol, or nalbufine.

Examples of therapeutic agents that are antiallergic compounds include amlexanox, astemizole, azelastinep, emirolast, alopatadine, cromolyn, fenpiprane, repirinast, tranilast, and traxanox.

Examples of therapeutic agents that are antianginal agents include nifedipine, atenol, bepridil, carazolol and epanolol

Examples of therapeutic agents that are anti-inflammatory analgesic agents include acetaminophen, methyl salicylate, monoglycol salicylate, aspirin, mefenamic acid, flufenamic acid, indomethacin, diclofenac, alclofenac, diclofenac sodium, ibuprofen, ketoprofen, naproxen, pranoprofen, fenoprofen, sulindac, fenclofenac, clidanac, flurbiprofen, fentiazac, bufexamac, piroxicam, phenylbutazone, oxyphenbutazone, clofezone, pentazocine, mepirizole, tiaramide hydrochloride, etc.

Examples of therapeutic agents that are steroidal anti-inflammatory agents include hydrocortisone, predonisolone, dexamethasone, triamcinolone acetonide, fluocinolone acetonide, hydrocortisone acetate, predonisolone acetate, methylpredonisolone, dexamethasone acetate, betamethasone, betamethasone valerate, flumetasone, fluorometholone, beclomethasone diproprionate, etc.

Examples of therapeutic agents that are antihistamines include diphenhydramine hydrochloride, diphenhydramine salicylate, diphenhydramine, chlorpheniramine hydrochloride, chlorpheniramine maleate isothipendyl hydrochloride, tripelennamine hydrochloride, promethazine hydrochloride, methdilazine hydrochloride, etc.

Examples of therapeutic agents that are vasoconstrictors include naphazoline nitrate, tetrahydrozoline hydrochloride, oxymetazoline hydrochloride, phenylephrine hydrochloride, tramazoline hydrochloride, etc.

Examples of therapeutic agents that are hemostatics include thrombin, phytonadione, protamine sulfate, aminocaproic acid, tranexamic acid, carbazochrome, carbaxochrome sodium sulfanate, rutin, hesperidin, etc.

Examples of therapeutic agents that are chemotherapeutic drugs include sulfamine, sulfathiazole, sulfadiazine, homosulfamine, sulfisoxazole, sulfisomidine, sulfamethizole, nitro furazone, taxanes, platinum compounds, topoisomerase I inhibitors, and anthrocycline.

Examples of therapeutic agents that are antibiotics include penicillin, meticillin, oxacillin, cefalotin, cefalordin, erythromycin, lincomycin, tetracycline, chlortetracycline, oxytetracycline, metacycline, chloramphenicol, kanamycin, streptomycin, gentamicin, bacitracin, cycloserine, and clindamycin.

Examples of therapeutic agents that are keratolytics include salicylic acid, podophyllum resin, podolifox, and cantharidin.

Examples of therapeutic agents that are growth factors include Autocrine motility factor, Bone morphogenetic proteins (BMP5), Epidermal growth factor (EGF), Erythropoietin (EPO), Fibroblast growth factor (FGF), Granulocyte-colony stimulating factor (G-CSF), Granulocyte-macrophage colony stimulating factor (GM-CSF), Growth differentiation factor-9 (GDF9), Hepatocyte growth factor (HGF), Hepatoma derived growth factor (HDGF), Insulin-like growth factor (IGF), migration-stimulating factor, Myostatin (GDF-8), Nerve growth factor (NGF) and other neurotrophins, Platelet-derived growth factor (PDGF), Thrombopoietin (TPO), Transforming growth factor alpha, Transforming growth factor beta (TGF-β), Vascular endothelial growth factor (VEGF), placental growth factor (P1GF) and Foetal Bovine Somatotrophin (FBS).

Examples of therapeutic agents that are growth hormone inhibitors are octreotide and somatostatin.

Examples of therapeutic agents that are hormones include Adiponectin, Adrenocorticotropic hormone (or corticotropin), Aldosterone, Androstenedione, Angiotensinogen and angiotensin, Antidiuretic hormone (or vasopressin, arginine vasopressin), Antimullerian hormone (or mullerian inhibiting factor or hormone), Atrial-natriuretic peptide (or atriopeptin), Brain natriuretic peptide, Calcidiol (25-hydroxyvitamin D3), Calcitonin, Calcitriol, Cholecystokinin, Corticotropin-releasing hormone, Cortisol, Dehydroepiandrosterone, Dihydrotestosterone, Dopamine (or prolactin inhibiting hormone), Endothelin, Enkephalin, Epinephrine (or adrenaline), Erythropoietin, Estradiol, Estriol, Estrone, Follicle-stimulating hormone, Gastrin, Ghrelin, Glucagon, Gonadotropin-releasing hormone, Growth hormone-releasing hormone, Histamine, Human chorionic gonadotropin, Human Growth hormone, Human placental lactogen, Inhibin, Insulin, Insulin-like growth factor (or somatomedin), Leptin, Leukotrienes, Lipotropin, Luteinizing hormone, Melanocyte stimulating hormone, Melatonin, Neuropeptide Y, Norepinephrine (or noradrenaline), Orexin, Oxytocin, Pancreatic polypeptide, Parathyroid hormone, Progesterone, Prolactin, Prolactin releasing hormone, Prostacyclin, Prostaglandins, Relaxin, Renin, Secretin, Serotonin, Somatostatin, Testosterone, Thrombopoietin, Thromboxane, Thyroid-stimulating hormone (or thyrotropin), Thyrotropin-releasing hormone, Thyroxine, Triiodothyronine.

Examples of therapeutic agents that are analgesic narcotics include fentanyl, buprenorphine, codeine sulfate, levorphanol, and morphine hydrochloride.

Examples of therapeutic agents that are antiviral drugs include Abacavir, Aciclovir, Acyclovir, Adefovir, Amantadine, Amprenavir, Ampligen, Arbidol, Atazanavir, Atripla, Boceprevir, Cidofovir, Combivir, Darunavir, Delavirdine, Didanosine, Docosanol, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Immunovir, Idoxuridine, Imiquimod, Indinavir, Inosine, Interferon type III, Interferon type II, Interferon type I, Interferon, Lamivudine, Lopinavir, Loviride, Maraviroc, Moroxydine, Nelfinavir, Nevirapine, Nexavir, Oseltamivir, Peginterferon alfa-2a, Penciclovir, Peramivir, Pleconaril, Podophyllotoxin, Raltegravir, Reverse transcriptase inhibitor, Ribavirin, Rimantadine, Ritonavir, Pyramidine, Saquinavir, Stavudine, Tea tree oil, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine

Examples of therapeutic agents that are drugs for the treatment of diabetes or its side effects includes insulin (natural or recombinant; monomer, hexamer, or mixtures thereof), insulin isophane, insulin lispro, insulin glargine, tolbutamide, acetohexamide, tolazamide, chlorpropamide, glipizide, glyburide, glimepiride, gliclazide, repaglinide, nateglinide, metformin, phenformin, buformin, rosiglitazone, pioglitazone, troglitazone, miglitol, acarbose, Glucagon-like peptide-1, Exanatide, Liraglutide, Taspoglutide, Lixisenatide, Albiglutide, vildagliptin, sitagliptin, saxagliptin, pramlintide, muraglitazar, tesaglitazar, aleglitazar

Examples of therapeutic agents that are drugs used for the treatment of CNS disorders include memantine hydrochloride, donepezil hydrochloride, rivastigmine tartrate, galantamine hydrochloride, tacrine hydrochloride.

Examples of therapeutic agents that are drugs used for the treatment of prostate cancer include Dutasteride, Bicalutamide, Ciprofloxacin, Erythromycin, Tamsulosin, Ofloxacin, Terazosin, Leuprolide, Nilutamide, Finasteride, Goserelin,

Examples of therapeutic agents that are drugs used for the treatment of ovarian cancer include Cisplatin, Carboplatin, Paclitaxel, Melphalan, Doxorubicin, hexamethylmelamine, Toptecan, Ifosfamide, Etoposide, 5-fluorouracil

Examples of therapeutic agents that are drugs used for the treatment of colorectal cancer include fluorouracil, bevacizumab, irinotecan, oxaliplatin, cetuximab, panitumumab, leucovorin, capecitabine.

Examples of therapeutic agents that are drugs used for the treatment of lung cancer include Carboplatin, Cisplatin, Docetaxel, Erlotinib, Etoposide, Gemcitabine, Gefitinib. Irinotecan, Paclitaxel, Pemetrexed, Topotecan, Vinorelbine, Gefitinib, Bevacizumab,

Examples of therapeutic agents that are drugs used for the treatment of melanoma include dacarbazine, interferon alfa-2b, aldesleukin, acarbazine.

Examples of therapeutic agents that are drugs used for the treatment multiple sclerosis include Interferon Beta 1a, Glatiramer Acetate, Mitoxantrone, Azathioprine, Cyclophosphamide, Cyclosporine, Methotrexate, Cladribine, MethylPrednisolone, Prednisone, Prednisolone, Dexamethasone, Corticotropin, Carbamazepine, Gabapentin, Topiramate, Zonisamide, Phenyloin, Desipramine, Amitriptyline, Imipramine

Examples of therapeutic agents that are drugs used for the treatment of Alzheimers disease include donepezil, galantamine, rivastigmine, memantine.

Examples of therapeutic agents that are drugs used for the treatment of arthritis include etanercept, infliximab, adalimumab, celecoxib, Rituximab, abatacept, etoricoxib, golimumab, ofatumumab, certolizumab pegol.

Examples of therapeutic agents that are drugs used for the treatment of blood deficiencies include pegfilgrastim, GCSF, PEG-GCSF, Darbepoetin alfa, Epoetin, Heparin (including low molecular weight derivatives), warfarin.

Examples of therapeutic agents that are drugs used for the treatment of mucositis include Palifermin.

Examples of protein therapeutic agents are also monoclonal antibodies, a polyclonal antibodies, humanized antibodies, antibody fragments, and immunoglobins.

Examples of therapeutic agents that are beneficial for RNA interference include, but are not limited to siRNA, dsDNA, miRNA, and antisense RNA.

Examples of therapeutic agents that are antibodies or their fragments include Abciximab, Adalimumab, Alemtuzumab, Basiliximab, Bevacizumab, Cetuximab, Certolizumab, Daclizumab, Eculizumab, Efalizumab, Gemtuzumab, Ibritumomab tiuxetan, Infliximab, Muromonab-CD3, Natalizumab, Omalizumab, Palivizumab, Panitumumab, Ranibizumab, Rituximab, Tositumomab, Trastuzumab,

Examples of therapeutic agents that are PEGylated drugs include Peginterferon alfa-2a, Peginterferon alfa-2b, Pegaspargase, and Pegfilgrastim.

Examples of therapeutic agents that are small molecules include Atorvastatin, Clopidrogel, Aripiprazole, Esomeprazole, Olanzapine, Quetiapine, Rosuvastatin, Montelukast, Venlafaxine Enoxaparin, and Pioglitazone.

### Compositions and Formulations

In another aspect, the present technology provides compositions comprising or consisting essentially of a nanoparticle of the present technology and a carrier, diluent, or excipient. In another embodiment, the carrier, diluent, or excipient is pharmaceutically acceptable. A variety of carrier, diluent, or excipient, pharmaceutically acceptable or not, are well known to one skilled in the art.

The nanoparticle may comprise an agent or agents which in turn are compounds or isomers, prodrug, tautomer, or pharmaceutically acceptable salts thereof, of the present technology can be formulated in the pharmaceutically acceptable compositions per se, or in the form of a hydrate, solvate, N-oxide, or pharmaceutically acceptable salt, as described herein. Typically, such salts are more soluble in aqueous solutions than the corresponding free acids and bases, but salts having lower solubility than the corresponding free acids and bases may also be formed. The present technology includes within its scope solvates of the compounds and salts thereof, for example, hydrates.

In one embodiment, the present technology provides a pharmaceutically acceptable composition (formulation) comprising a nanoparticle and at least one pharmaceutically acceptable excipient, diluent, preservative, stabilizer, or mixture thereof.

In one embodiment, the methods can be practiced as a therapeutic approach towards the treatment of the conditions described herein. Thus, in a specific embodiment, the compounds of the present technology can be used to treat the conditions described herein in animal subjects, including humans. The methods generally comprise administering to the subject a nanoparticle of the present technology, or a salt, prodrug, hydrate, or N-oxide thereof, effective to treat the condition. As used herein, prodrug of a compound of the present technology is a compound that is converted in vivo or in vitro to the compound of the present technology. Hydrolysis, oxidation, and/or reduction are some ways that a prodrug is converted to the compound of the present technology.

In some embodiments, the subject is a non-human mammal, including, but not limited to, bovine, horse, feline, canine, rodent, or primate. In another embodiment, the subject is a human.

The nanoparticles of the present technology can be provided in a variety of formulations and dosages. It is to be understood that reference to the compound of the present technology, or “active” in discussions of formulations is also intended to include, where appropriate as known to those of skill in the art, formulation of the salts and prodrugs of the compounds.

Pharmaceutically acceptable compositions comprising the nanoparticles described herein (or salts or prodrugs thereof) can be manufactured by means of conventional mixing, dissolving, granulating, dragee-making, levigating, emulsifying, encapsulating, entrapping, or lyophilization processes. The compositions can be formulated in conventional manner using one or more physiologically acceptable carriers, diluents, excipients, or auxiliaries which facilitate processing of the active compounds into preparations which can be used pharmaceutically.

The nanoparticles of the present technology can be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration.

The pharmaceutically acceptable compositions for the administration of the compounds can be conveniently presented in unit dosage form and can be prepared by any of the methods well known in the art. The pharmaceutically acceptable compositions can be, for example, prepared by uniformly and intimately bringing the active ingredient into association with a liquid carrier, a finely divided solid carrier or both, and then, if necessary, shaping the product into the desired formulation. In the pharmaceutical composition the active object compound is included in an amount sufficient to produce the desired therapeutic effect. For example, pharmaceutically acceptable compositions of the present technology may take a form suitable for virtually any mode of administration, including, for example, topical, ocular, oral, buccal, systemic, nasal, injection, transdermal, rectal, and vaginal, or a form suitable for administration by inhalation or insufflation.

For topical administration, the compound(s), salt(s) or prodrug(s) can be formulated as solutions, gels, ointments, creams, suspensions, etc., as is well-known in the art.

Systemic pharmaceutically acceptable compositions include those designed for administration by injection (e.g., subcutaneous, intravenous, intramuscular, intrathecal, or intraperitoneal injection) as well as those designed for transdermal, transmucosal, oral, or pulmonary administration.

Useful injectable pharmaceutically acceptable compositions include sterile suspensions, solutions, or emulsions of the active compound(s) in aqueous or oily vehicles. The pharmaceutically acceptable compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable pharmaceutically acceptable compositions can be provided in powder form for reconstitution with a suitable vehicle, including but not limited to sterile pyrogen free water, buffer, and dextrose solution, before use. To this end, the active compound(s) can be dried by any art-known technique, such as lyophilization, and reconstituted prior to use.

For transmucosal administration, penetrants appropriate to the barrier to be permeated are used in the pharmaceutically acceptable compositions. Such penetrants are known in the art.

For oral administration, the pharmaceutically acceptable compositions may take the form of, for example, lozenges, tablets, or capsules prepared by conventional means with pharmaceutically acceptable excipients such as binding agents (e.g., pregelatinized maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings. Additionally, the pharmaceutically acceptable compositions containing the compounds of the present technology or prodrug thereof in a form suitable for oral use may also include, for example, troches, lozenges, aqueous, or oily suspensions, dispersible powders or granules, emulsions, hard or soft capsules, or syrups or elixirs.

Pharmaceutically acceptable compositions intended for oral use can be prepared according to any method known to the art for the manufacture of pharmaceutically acceptable compositions, and such compositions may contain one or more agents selected from the group consisting of sweetening agents, flavoring agents, coloring agents, and preserving agents in order to provide pharmaceutically elegant and palatable preparations. Tablets contain the active ingredient (including drug and/or prodrug) in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in the U.S. Pat. Nos. 4,256,108; 4,166,452; and 4,265,874 to form osmotic therapeutic tablets for control release. The pharmaceutically acceptable compositions of the present technology may also be in the form of oil-in-water emulsions.

Liquid pharmaceutically acceptable compositions (or liquid preparations) for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); nonaqueous vehicles (e.g., almond oil, oily esters, ethyl alcohol, Cremophore™, or fractionated vegetable oils); and preservatives (e.g., methyl or propylhydroxybenzoates or sorbic acid). The preparations may also contain buffer salts, preservatives, flavoring, coloring, and sweetening agents as appropriate.

Preparations for oral administration can be suitably formulated to give controlled release or sustained release of the active compound, as is well known. The sustained release formulations (or sustained release pharmaceutically acceptable compositions) of the present technology are preferably in the form of a compressed tablet comprising an intimate mixture of compound of the present technology and a partially neutralized pH-dependent binder that controls the rate of compound dissolution in aqueous media across the range of pH in the stomach (typically approximately 2) and in the intestine (typically approximately about 5.5).

To provide for a sustained release of compounds of the present technology, one or more pH-dependent binders can be chosen to control the dissolution profile of the sustained release pharmaceutically acceptable compositions so that such pharmaceutically acceptable compositions release compound slowly and continuously as the pharmaceutically acceptable compositions are passed through the stomach and gastrointestinal tract. Accordingly, the pH-dependent binders suitable for use in the present technology are those which inhibit rapid release of drug from a tablet during its residence in the stomach (where the pH is-below about 4.5), and which promotes the release of a therapeutic amount of the compound of the present technology from the dosage form in the lower gastrointestinal tract (where the pH is generally greater than about 4.5). Many materials known in the pharmaceutical art as “enteric” binders and coating agents have a desired pH dissolution properties. The examples include phthalic acid derivatives such as the phthalic acid derivatives of vinyl polymers and copolymers, hydroxyalkylcelluloses, alkylcelluloses, cellulose acetates, hydroxyalkylcellulose acetates, cellulose ethers, alkylcellulose acetates, and the partial esters thereof, and polymers and copolymers of lower alkyl acrylic acids and lower alkyl acrylates, and the partial esters thereof. One or more pH-dependent binders present in the sustained release formulation of the present technology are in an amount ranging from about 1 to about 30 wt %, about 5 to about 12 wt % and about 10 wt %.

One or more pH-independent binders may be in used in oral sustained release pharmaceutically acceptable compositions of the present technology. The pH-independent binders can be present in the pharmaceutically acceptable compositions of the present technology in an amount ranging from about 1 to about 10 wt %, from about 1 to about 3 wt % and about 2 wt %.

The sustained release pharmaceutically acceptable compositions of the present technology may also contain pharmaceutically acceptable excipients intimately admixed with the compound and the pH-dependent binder. Pharmaceutically acceptable excipients may include, for example, pH-independent binders or film-forming agents such as hydroxypropyl methylcellulose, hydroxypropyl cellulose, methylcellulose, polyvinylpyrrolidone, neutral poly(meth)acrylate esters, starch, gelatin, sugars, carboxymethylcellulose, and the like. Other useful pharmaceutical excipients include diluents such as lactose, mannitol, dry starch, microcrystalline cellulose and the like; surface active agents such as polyoxyethylene sorbitan esters, sorbitan esters and the like; and coloring agents and flavoring agents. Lubricants (such as talc and magnesium stearate) and other tableting aids can also be optionally present.

The sustained release pharmaceutically acceptable compositions of the present technology have a compound of the present technology in the range of about 50% by weight to about 95% or more by weight, about 70% to about 90% by weight; a pH-dependent binder content of between 5% and 40%, between 5% and 25%, and between 5% and 15%; with the remainder of the dosage form comprising pH-independent binders, fillers, and other optional excipients.

For buccal administration, the pharmaceutically acceptable compositions may take the form of tablets or lozenges formulated in the conventional manner.

For rectal and vaginal routes of administration, the active compound(s) can be formulated as solutions (for retention enemas), suppositories, or ointments containing conventional suppository bases such as cocoa butter or other glycerides.

For nasal administration or administration by inhalation or insufflation, the active compound(s) or prodrug(s) can be conveniently delivered in the form of an aerosol spray from pressurized packs or a nebulizer with the use of a suitable propellant (e.g., dichlorodifluoromethane, trichlorofluoromethane, dichlorotetrafluoroethane, fluorocarbons, carbon dioxide, or other suitable gas). In the case of a pressurized aerosol, the dosage unit can be determined by providing a valve to deliver a metered amount. Capsules and cartridges for use in an inhaler or insufflator (for example, capsules and cartridges comprised of gelatin) can be formulated containing a powder mix of the compound and a suitable powder base such as lactose or starch.

The pharmaceutically acceptable compositions can be in the form of a sterile injectable aqueous or oleaginous suspension. This suspension can be formulated according to the known art using those suitable dispersing or wetting agents and suspending agents which have been mentioned above. The sterile injectable preparation may also be a sterile injectable solution or suspension in a non-toxic parenterally acceptable diluent or solvent. Among the acceptable vehicles and solvents that can be employed are water, Ringer's solution, and isotonic sodium chloride solution. The compounds may also be administered in the form of suppositories for rectal or urethral administration of the drug.

For topical use, creams, ointments, jellies, gels, solutions, suspensions, etc., containing the nanoparticles of the present technology, can be employed. In some embodiments, the compounds of the present technology can be formulated for topical administration with polyethylene glycol (PEG). These formulations may optionally comprise additional pharmaceutically acceptable ingredients such as diluents, stabilizers, and/or adjuvants.

Included among the devices which can be used to administer nanoparticles of the present technology, are those well-known in the art, such as metered dose inhalers, liquid nebulizers, dry powder inhalers, sprayers, thermal vaporizers, and the like. Other suitable technology for administration of particular nanoparticles of the present technology includes electrohydrodynamic aerosolizers. As those skilled in the art will recognize, the formulation of nanoparticles, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of nanoparticles in the aerosol. For example, shorter periods of administration can be used at higher concentrations of nanoparticles in the nebulizer solution. Devices such as metered dose inhalers can produce higher aerosol concentrations and can be operated for shorter periods to deliver the desired amount of nanoparticles in some embodiments. Devices such as dry powder inhalers deliver active agent until a given charge of agent is expelled from the device. In this type of inhaler, the amount of nanoparticles in a given quantity of the powder determines the dose delivered in a single administration.

Pharmaceutically acceptable compositions of the nanoparticles of the present technology for administration from a dry powder inhaler may typically include a finely divided dry powder containing nanoparticles, but the powder can also include a bulking agent, buffer, carrier, excipient, another additive, or the like. Additives can be included in such a dry powder composition of nanoparticles of the present technology, for example, to dilute the powder as required for delivery from the particular powder inhaler, to facilitate processing of the formulation, to provide advantageous powder properties to the formulation, to facilitate dispersion of the powder from the inhalation device, to stabilize the formulation (e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; and the like.

For prolonged delivery, the nanoparticle(s) or prodrug(s) of the present technology can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives (e.g., as a sparingly soluble salt). Alternatively, transdermal delivery systems manufactured as an adhesive disc or patch which slowly releases the active nanoparticle(s) for percutaneous absorption can be used. To this end, permeation enhancers can be used to facilitate transdermal penetration of the active nanoparticle(s). Suitable transdermal patches are described in, for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems can be employed. Liposomes and emulsions are well-known examples of delivery vehicles that can be used to deliver active nanoparticle(s) or prodrug(s). Certain organic solvents such as dimethylsulfoxide (DMSO) may also be employed, for example for topical administration, although usually at the cost of greater toxicity.

The pharmaceutical compositions may, if desired, be presented in a pack or dispenser device which may contain one or more unit dosage forms containing the active nanoparticle(s). The pack may, for example, comprise metal or plastic foil, such as a blister pack. The pack or dispenser device can be accompanied by instructions for administration.

The nanoparticles described herein, or compositions thereof, will generally be used in an amount effective to achieve the intended result, for example, in an amount effective to treat or prevent the particular condition being treated. The nanoparticles can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also includes halting or slowing the progression of the disease, regardless of whether improvement is realized.

The amount of nanoparticle administered will depend upon a variety of factors, including, for example, the particular condition being treated, the mode of administration, the severity of the condition being treated, the age and weight of the patient, the bioavailability of the particular active nanoparticle. Determination of an effective dosage is well within the capabilities of those skilled in the art. As known by those of skill in the art, the preferred dosage of nanoparticles of the present technology will also depend on the age, weight, general health, and severity of the condition of the individual being treated. Dosage may also need to be tailored to the sex of the individual and/or the lung capacity of the individual, where administered by inhalation. Dosage, and frequency of administration of the nanoparticles or prodrugs thereof, will also depend on whether the nanoparticles are formulated for treatment of acute episodes of a condition or for the prophylactic treatment of a disorder. A skilled practitioner will be able to determine the optimal dose for a particular individual.

For prophylactic administration, the nanoparticle can be administered to a patient at risk of developing one of the previously described conditions. Alternatively, prophylactic administration can be applied to avoid the onset of symptoms in a patient diagnosed with the underlying disorder.

Effective dosages can be estimated initially from in vitro assays. For example, an initial dosage for use in animals can be formulated to achieve a circulating blood or serum concentration of active nanoparticle that is at or above an IC₅₀ of the particular nanoparticle as measured in as in vitro assay. Calculating dosages to achieve such circulating blood or serum concentrations taking into account the bioavailability of the particular nanoparticle is well within the capabilities of skilled artisans. For guidance, the reader is referred to Fingl & Woodbury, “General Principles,” GOODMAN AND GILMAN′S THE PHARMACEUTICAL BASIS OF THERAPEUTICS, Chapter 1, pp. 1-46, latest edition, Pergamon Press, and the references cited therein.

Initial dosages can also be estimated from in vivo data, such as animal models. Certain animal models useful for testing the efficacy of nanoparticles to treat or prevent the various diseases described above are well-known in the art. Ordinarily skilled artisans can routinely adapt such information to determine dosages suitable for human administration.

Dosage amounts will typically be in the range of from about 0.0001 or about 0.001 or about 0.01 mg/kg/day to about 100 mg/kg/day, but can be higher or lower, depending upon, among other factors, the activity of the nanoparticle, its bioavailability, the mode of administration, and various factors discussed above. Dosage amount and interval can be adjusted individually to provide levels in the organ system of interest of the nanoparticle(s) which are sufficient to maintain therapeutic or prophylactic effect. For example, the nanoparticles can be administered once per week, several times per week (e.g., every other day), once per day, or multiple times per day, depending upon, among other things, the mode of administration, the specific indication being treated, and the judgment of the prescribing physician. In cases of local administration or selective uptake, such as local topical administration, the effective local concentration of active nanoparticle(s) may not be related to plasma concentration. Skilled artisans will be able to optimize effective local dosages without undue experimentation.

The nanoparticle(s) useful in the treatment methods of the present technology will provide therapeutic or prophylactic benefit without causing substantial toxicity. Toxicity of the nanoparticle(s) can be determined using standard pharmaceutical procedures. The dose ratio between toxic and therapeutic (or prophylactic) effect is the therapeutic index. In certain embodiments, the nanoparticles(s) exhibit high therapeutic indices as pertinent to the disease treated.

The foregoing disclosure pertaining to the dosage requirements for the nanoparticles of the present technology is pertinent to dosages required for prodrugs, with the realization, apparent to the skilled artisan, that the amount of prodrug(s) administered will also depend upon a variety of factors, including, for example, the bioavailability of the particular prodrug(s) and the conversation rate and efficiency into active drug nanoparticle under the selected route of administration. Determination of an effective dosage of prodrug(s) for a particular use and mode of administration is well within the capabilities of those skilled in the art.

### Kits

Also provided are kits for administration of the nanoparticles of the present technology or pharmaceutical formulations comprising the nanoparticle that may include a dosage amount of at least one nanoparticle or a composition comprising at least one nanoparticle, as disclosed herein. Kits may further comprise suitable packaging and/or instructions for use of the nanoparticle. Kits may also comprise a means for the delivery of the at least one nanoparticle or compositions comprising at least one nanoparticle of the present technology, such as an inhaler, spray dispenser (e.g., nasal spray), syringe for injection, or pressure pack for capsules, tablets, suppositories, or other device as described herein.

Other types of kits provide the nanoparticle and reagents to prepare a composition of the present technology for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch, or inhalant.

The kits may include other therapeutic nanoparticles or therapeutic agents for use in conjunction with the nanoparticles of the present technology described herein. These nanoparticles can be provided in a separate form or mixed with the nanoparticles of the present technology. The kits will include appropriate instructions for preparation and administration of the composition, side effects of the compositions, and any other relevant information. The instructions can be in any suitable format, including, but not limited to, printed matter, videotape, computer readable disk, or optical disc.

In one embodiment, the present technology provides a kit comprising a nanoparticle, micelle or liposome as described herein, packaging, and instructions for use.

In another embodiment, the present technology provides a kit comprising the pharmaceutically acceptable composition comprising a nanoparticle, micelle or liposome as described herein and at least one pharmaceutically acceptable excipient, diluent, preservative, stabilizer, or mixture thereof, packaging, and instructions for use. In another embodiment, kits for treating an individual who suffers from or is susceptible to the conditions described herein are provided, comprising a container comprising a dosage amount of a nanoparticle, micelle, liposome or composition of the present technology, as disclosed herein, and instructions for use. The container can be any of those known in the art and appropriate for storage and delivery of oral, intravenous, topical, rectal, urethral, or inhaled formulations.

Kits may also be provided that contain sufficient dosages of the nanoparticles or composition to provide effective treatment for an individual for an extended period, such as a week, 2 weeks, 3, weeks, 4 weeks, 6 weeks, or 8 weeks or more.

The technology having been described in summary and in detail is illustrated and not limited by the examples below.

### EXPERIMENTAL EXAMPLES

### Example 1

### Synthesis of Dextran Succinate (DS)

70 kDa Dextran (10 g) was stirred in dry dimethylsulfoxide (100 mL) and pyridine (15 mL). Succinic anhydride (1.54 g) was added and the mixture, which became a homogenous solution after 1 hour, was stirred at room temperature under argon for 16 hours. The solution was poured into stirred ethyl acetate (400 mL), and then acetone (400 mL) was added and stirring was continued for 16 hours, during which the pasty precipitate eventually became granular. The precipitate was filtered, washed with ethyl acetate and dried under vacuum to afford a white solid, which was dissolved in water (250 mL). The aqueous solution was acidified with dilute HCl to pH 2 and 5× diafiltered with water using a 0.1 m² TFF (tangential flow filtration) module with a 5 kDa MWCO membrane. The solution was then concentrated to ˜50 mL by TFF and lyophilized to afford dextran 20% succinate as a white solid (10.2 g). ¹H NMR analysis confirmed that the product contained 0.2 equivalents of succinate per anhydroglucose unit (20% succinylation).

### Example 2

### Synthesis of 70 kDa VB12-Dextran Succinate Conjugate

70 kDa Dextran succinate of Example 1 (200 mg) and aminohexyl-VB12 (20 mg; J F McEwan et al, Bioconjugate Chem. 1999, 10, 1131-1136) were dissolved in water (8 mL). 1-Ethyl-3-(3-dimethylaminopropyl) carbodiimide hydrochloride (200 mg) and N hydroxysuccinimide (200 mg) were added and the solution (pH 5.5) was stirred for 16 hours. The mixture was centrifuged in a 5 kDa Amicon 15 centrifugal filter at 3800 rpm for 45 min. Water (15 mL) was added to the retentate and centrifuged; then the 15 mL wash was repeated once more. The washed retentate was lyophilized to afford Cob-DS (223 mg) as a pale red solid. UV-VIS spectrophotometric analysis revealed the product contained 3.25% w/w of VB12, which corresponds to ˜0.5 equivalents of AH-VB12 per 100 anhydroglucose units (0.5 mol % VB12).

### Example 3

### Synthesis of 70 kDa Carboxymethyl Dextran

A solution of 70 kDa dextran (4.0 g) in 11% sodium hydroxide (20 mL) was added to a solution of chloroacetic acid (2.3 g) in tert butanol (40 mL) and the biphasic mixture was stirred vigorously at 60° C. for 3 hours. After cooling to room temperature, the mixture was poured into stirring acetone (400 mL) and the resulting pasty precipitate was separated by decantation. The paste was dissolved in water (25 mL) and poured into stirring methanol (300 mL) and the resulting white precipitate was filtered, washed with methanol and dried under vacuum. The crude product was dissolved in water and 5× diafiltered with water using a 0.1 m2 TFF (tangential flow filtration) module with a 5 kDa MWCO membrane. The solution was then concentrated by TFF and lyophilized to afford a white solid (4.6 g). ¹H NMR analysis revealed that the product contained 0.2 carboxy-methyl equivalents per anhydroglucose unit (20% carboxymethylation).

### Example 4

### Synthesis of 2000 kDa Carboxymethyl Dextran

To a solution of 2000 kDa dextran (2.0 g; made in a manner similar to that described in Example 1) in water (20 mL) and sodium hydroxide (1.7 g) was added a solution of chloroacetic acid (2.3 g) in tert butanol (40 mL) and the biphasic mixture was stirred vigorously at 60° C. for 6 hours. After cooling to room temperature, the mixture was adjusted to pH 5 with HCl, then poured into stirring methanol (200 mL) and the resulting white precipitate was separated by centrifugation. The precipitate was washed twice with methanol (2×200 mL) by centrifugation and dried under vacuum overnight to afford a white solid (2.5 g). ¹H NMR analysis revealed that the product contained 0.26 carboxymethyl equivalents per anhydroglucose unit (26% carboxymethylation).

### Example 5

### Synthesis of 70 kDa VB12-Aminoethylamido-Carboxymethyl Dextran Conjugate

20% Carboxymethyl 70 kDa dextran (200 mg) and aminohexyl-VB12 (75 mg) were dissolved in water (10 mL). 1-Ethyl-3-(3-dimethylaminopropyl) carbodiimide hydrochloride (EDAC; 60 mg) and N hydroxysuccinimide (NHS; 15 mg) were added and the solution was stirred for 4 hours at pH 5.3-5.7. Ethylenediamine dihydrochloride (164 mg) and a further portion of EDAC (176 mg) were added, the pH was adjusted to pH 5.6 and the reaction was stirred overnight. The mixture was centrifuged in a 5 kDa Amicon 15 centrifugal filter at 3800 rpm for 30 min. Water (15 mL) was added to the retentate and centrifuged, and then the 15 mL wash was repeated twice more. The retentate was lyophilized to afford Cob-EDCMD70 (170 mg) as a pale red solid. UV-VIS spectrophotometric analysis revealed the product contained 6.5% w/w of VB12, which corresponds to 0.9 equivalents of VB12 per 100 anhydroglucose units (0.9 mol % VB12).

### Example 6

### Synthesis of 2000 kDa VB12-Carboxymethyl Dextran Conjugate

26% Carboxymethyl 2000 kDa dextran (200 mg) and aminohexyl-VB12 (100 mg) were dissolved in water (10 mL). 1-Ethyl-3-(3-dimethylaminopropyl) carbodiimide hydrochloride (EDAC; 30 mg) and N hydroxysuccinimide (NHS; 15 mg) were added and the solution was stirred for 50 hours at pH 4.9-5.6. The mixture was centrifuged in a 5 kDa Amicon 15 centrifugal filter at 3800 rpm for 30 min. Water (15 mL) was added to the retentate and centrifuged, and then the 15 mL water wash was repeated six times more. The retentate was lyophilized to afford Cob-CMD2K (198 mg) as a pale red solid. UV-VIS spectrophotometric analysis revealed the product contained 17.5% w/w of VB12, which corresponds to ˜2.7 equivalents of VB12 per 100 anhydroglucose units (2.7 mol % VB12).

### Example 7

### Synthesis of 2000 kDa VB12-Aminohexamethylamido-Carboxymethyl Dextran Conjugate

26% Carboxymethyl 2000 kDa dextran (200 mg) and aminohexyl-VB12 (50 mg) were dissolved in water (10 mL). 1-Ethyl-3-(3-dimethylaminopropyl) carbodiimide hydrochloride (EDAC; 30 mg) and N hydroxysuccinimide (NHS; 15 mg) were added and the solution was stirred for 5 hours at pH 5.0-5.7. Hexamethylenediamine dihydrochloride (210 mg) and a further portion of EDAC (173 mg) were added, the pH was adjusted to pH 5.2 and the reaction was stirred overnight. The mixture was centrifuged in a 5 kDa Amicon 15 centrifugal filter at 3800 rpm for 30 min. Water (15 mL) was added to the retentate and centrifuged, and then the 15 mL water wash was repeated until the filtrate was colorless. The retentate was lyophilized to afford Cob-AHCMD2K (156 mg) as a pale red solid. UV-VIS spectrophotometric analysis revealed the product contained 2.3% w/w of VB12, which corresponds to ˜0.3 equivalents of VB12 per 100 anhydroglucose units (0.3 mol % VB12).

### Example 8

### Synthesis of VB12-Abraxane Conjugate

Abraxane (900 mg) was shaken with water (50 mL) and the pH of the milky white suspension was adjusted to pH 6.0. Aminohexyl-VB12 (200 mg) and N hydroxysuccinimide (NHS; 43 mg) were added and the pH was adjusted to pH 5.3. 1-Ethyl-3-(3-dimethylaminopropyl) carbodiimide hydrochloride (EDAC; 71 mg) was added and the suspension was stirred for 15 hours at pH 5.3-5.6. The mixture was 10× diafiltered with water using a Millipore Pellicon XL 5 kDa Biomax 50 cm2 filter cassette, then concentrated to ˜15 mL. The retentate was lyophilized to afford Cob-Abraxane (883 mg) as a pale red solid. Cobalt analysis by ICP revealed the product contained 678 ppm of cobalt, which corresponds to 1.56% w/w of VB12.

### Example 9

### Preparation of Insulin Nanoparticles

The VB12 derivative of Example 5 (47.5 mg) was added to a solution of bovine insulin in dilute HCl (5.0 mg/mL; 0.5 mL) and agitated gently for 1 hour at room temperature, then lyophilized to afford insulin nanoparticles as a pale red solid. Reconstitution of the nanoparticles in water (14.4 mg per mL) provided an insulin dosage of 20 IU per mL.

### Example 10

### Blood Glucose Reduction in Diabetic Rats with Insulin Nanoparticles

Female Wistar rats (200 g) were housed at room temperature with 12 h light/dark cycle. All animals had ad libitum access to a standard chow diet and water except wherever indicated. The rats were allowed to acclimatize for a period of 7 days in the new environment before initiation of the experiment. After randomization into groups of 4, each rat was marked and followed individually throughout the study. All rats were fasted for 1 hour. Streptozotocin obtained from Sigma (98% HPLC) was administered by IV injection at a dose of 55-65 mg/kg in 0.1M citrate buffer (pH 4.5) followed by an additional 1 hour fast. Blood was collected daily from the tail and blood glucose levels were measured using an Accu-Chek® (Compact Plus-Roche) blood glucose monitor. When all animals in the group had achieved a blood glucose level of >250 mg/dl (5 days) the insulin metabolism phase was begun. All animals were fasted for 1 hour and a blood glucose measurement taken (T=0). Groups of rats were dosed by oral gavage with 0.5 mL of an aqueous preparation of insulin nanoparticles (containing 20 IU/mL of insulin) of example 9, or 0.5 mL of an aqueous solution of bovine insulin (20 IU/mL). The animals were then fasted for another hour and blood glucose levels were measured at 1, 4, 8 and 24 hours after dosing. Administration of the insulin nanoparticle formulation resulted in reductions (compared to T=0) of blood glucose levels of 28% at 8 hours and 12% at 24 hours, while the plain insulin formulation resulted in increases in blood glucose levels of 1% at 8 hours and 21% at 24 hours. The results show a significant reduction in blood glucose by insulin nanoparticles compared to oral administration of unformulated insulin.

### Example 11

### Inhibition of Tumor Growth with Abraxane or Cobrazane Conjugates

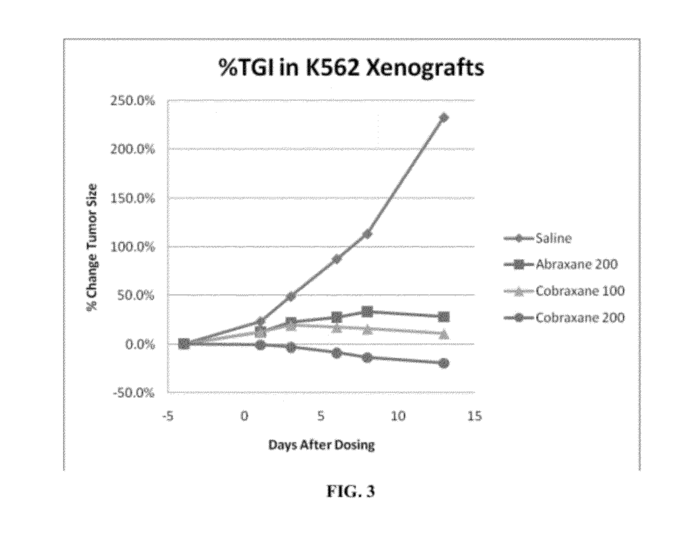

Athymic nude mice were implanted with human leukemia K562 cells and xenograft tumors allowed to grow until 150-200 mm³ in size. Animals were randomized into groups of seven and dosed by intraperitoneal injection with either saline control, Abraxane (200 mg/kg paclitaxel) or Cobraxane (100 or 200 mg/kg paclitaxel) and tumor sizes were measured three times per week. The plot in FIG. 3 shows inhibition of tumor growth (relative to saline control) for all three active groups. Cobraxane at 50% of paclitaxel dose was superior to Abraxane and an equivalent dose of Cobraxane actually reduced the tumor size.

### Example 12

### Synthesis of VB12-TriMethylChitosan (VB12-TMC)

Chitosan (Aldrich Low MW; 10 g) was suspended in water (180 mL) and formaldehyde (40 mL) and formic acid (30 mL) were added. The mixture was heated at 70° C. for 24 hours, evolving copious quantities of gas (CO₂). Further portions of formaldehyde (40 mL) and formic acid (30 mL) were added and the mixture heated at 70° C. for another 24 hours, at which time gas evolution had ceased completely. Water (200 mL) was added and the solution was filtered through Celite, then subjected to tangential flow filtration with a 5 kDa MWCO membrane, concentrated by TFF and lyophilized to afford N,N-dimethyl chitosan as a white solid (9.1 g). ¹H NMR analysis revealed that all of the non-acetylated amine groups of chitosan had been converted to dimethylamino groups. N,N-Dimethyl chitosan (6.2 g) was suspended in N-methyl pyrrolidone (200 mL) and the mixture was heated at 70° C. for 1 hr, then cooled. Methyl iodide (10 mL) was added, the mixture heated at 40° C. for 4 hours, more methyl iodide (10 mL) was added and the mixture maintained at 40° C. for 24 hours. More methyl iodide (5 mL) was added and the mixture maintained at 40° C. for a further 24 hours. The reaction was slowly added to ethyl acetate (600 mL) and the resulting solid was filtered, washed with ethyl acetate and dried to afford crude TMC iodide (8.5 g) as a brown solid. Sodium hydride 60% suspension (318 mg) was added to dry DMSO (75 mL) and the mixture was heated at 70° C. for 1 hour. After cooling, crude trimethyl chitosan iodide (2 g) was added and the mixture stirred at room temperature for 3 hours. Chloroacetic acid (250 mg) was added and the reaction stirred for 50 hours, then poured into stirring acetone (400 mL). The resulting precipitate was isolated by centrifugation, washed with acetone and dried to afford a white solid (˜2 g). The solid was dissolved in 1 M NaCl in 0.1 M HCl (100 mL), filtered, subjected to tangential flow filtration with a 5 kDa MWCO membrane, concentrated by TFF and lyophilized to afford O-carboxymethyl N,N,N-trimethyl chitosan (CMTMC) as a white solid (1.17 g). ¹³C NMR analysis confirmed the presence of carboxymethyl groups. O-Carboxymethyl N,N,N-trimethyl chitosan (500 mg) was dissolved in water (40 mL) and the solution adjusted to pH 5.3. Aminohexyl-VB12 (40 mg), EDAC (19 mg) and NHS (12 mg) were added and the solution was stirred for 2.5 hours. More EDAC (20 mg) was added and the mixture stirred for 16 hours. The solution was subjected to tangential flow filtration with a 5 kDa MWCO membrane, concentrated by TFF and lyophilized to afford VB12-Carboxymethyl-TriMethylChitosan; VB12-TMC (413 mg) as a pale red solid.

### Example 13

### Synthesis of VB12-PLGA

To a suspension of poly(lactic-co-glycolic acid) (PLGA RG 502H; 100 mg) and NHS (60 mg) in dichloromethane (10 mL) was added EDAC (100 mg) and the mixture was stirred for 18 hours at room temperature. The solution was evaporated to ˜3 mL, then added to diethyl ether (10 mL). The resulting solid was washed with ether and vacuum dried to afford PLGA NHS ester as a white solid (209 mg). To a solution of aminohexyl-VB12 (40 mg) in DMF (2 mL) was added triethylamine (6 drops), followed by a solution of PLGA NHS ester (209 mg) in DMF (5 mL). The solution was stirred for 16 hours, then added to cold ether (40 mL) and the resulting precipitate was centrifuged, washed with ether (2×20 mL) and dried under high vacuum to afford PLGA-amidohexyl-VB12 (VB12-PLGA) as a red solid (112 mg). Cobalt analysis by ICP revealed the product contained 6352 ppm of cobalt, which corresponds to 14.6% w/w of VB12.

### Example 14

### Synthesis of VB12-PLGA nanoparticles containing Insulin

A 7 mL vial was charged with human recombinant insulin (2.0 mg), RG 502H PLGA (37.5 mg) and VB12-PLGA (1.5 mg). A mixed solvent system of 2.3 mL acetone and 0.4 mL 10 mM HCl was then added with rapid shaking for 20 min. This solution was added to a stirring 30 mL volume of 10 mg/mL PVA solution (Mowiol 4-88) forming a pale pink suspension, with stirring for 1 h. The mixture was then centrifuged at 10,500 rpm for 30 min, the supernatant decanted and the pink pellet washed with deionized water (2×15 mL) and lyophilized to yield 30.9 mg of pink nanoparticles; Z-average=273 nm, PDI=0.266; Zeta potential=−28.1 mV.

### Example 15

### Synthesis of VB12-PLGA-HP55 Nanoparticles Containing Insulin

500 μL of 3 mg/mL VB12-PLGA solution in acetone was added to 1.2 mL of 25 mg/mL RG 502H PLGA solution also in acetone. This mixture was shaken for 5 min and 1.2 mL of 12.5 mg/mL hypromellose phthalate (HP-55) solution in acetone added, with shaking for another 5 min. 400 μL of 5 mg/mL recombinant human insulin solution in 10 mM HCL was then added to form a clear pink solution which was shaken for 10 min. This mixture was added to a rapidly stirring 30 mL volume of 10 mg/mL PVA solution (Mowiol 4-88) to produce a turbid pink suspension which was then stirred for 1 h. This was centrifuged at 10,500 rpm for 20 min, the supernatant decanted and the pink pellet washed with deionized water (2×15 mL), and lyophilized to yield 29.6 mg of pink nanoparticles; Z-average=294 nm, PDI=0.114; Zeta potential=−55.0 mV.

### Example 16

### Synthesis of VB12-PLGA-HP55 Nanoparticles Containing Insulin

A 7 mL vial was charged with human recombinant insulin (2.0 mg), RG 502H PLGA (37.5 mg), VB12-PLGA (1.5 mg) and HP-55 (7.5 mg). A mixed solvent system of 2.6 mL acetone and 0.4 mL 10 mM HCl was then added with rapid shaking for 20 min to produce a clear pink solution. This solution was added to a stirring 30 mL volume of 10 mg/mL PVA solution (Mowiol 4-88) forming a pale pink suspension, with stirring for 1 h. The mixture was then centrifuged at 10,500 rpm for 30 min, the supernatant decanted and the pink pellet washed with deionized water (2×15 mL) and lyophilized to yield 32.1 mg of pink nanoparticles; Z-average=245 nm, PDI=0.076; Zeta potential=−52.9 mV.

### Example 17

### Synthesis of VB12-TMC-PLGA Nanoparticles Containing siRNA

100 μL of 10 mg/mL siRNA solution was added to 200 μL of 10 mg/mL VB12-TMC solution and shaken for 5 min to produce a turbid suspension. This was added to 750 μL of 40 mg/mL Resomer RG 502H PLGA solution in dichloromethane and the mixture sonicated for 1 min. To this pink emulsion was added 2 mL of 20 mg/mL PVA (˜10,000 MW, 80% hydrolyzed) solution with further sonication for 1 min. The mixture was then added to 12 mL of 20 mg/mL PVA solution and with rapid stirring (uncapped) for 90 min. It was then centrifuged at 10,500 rpm for 20 min at 4° C. and the supernatant decanted. The pellet was washed with deionized water (2×5 mL) and then lyophilized to yield 21.1 mg of pink nanoparticles; Z-average=270 nm, PDI=0.268; zeta potential=−38.4 mV.

### Example 18

### Synthesis of VB12-Oleamide

To a suspension of oleic acid (1.0 g) and NHS (447 mg) in dichloromethane (40 mL) was added EDAC (743 mg) and the mixture was stirred for 20 hours at room temperature. The solution was washed with ice-cold water (3×40 mL), dried over Na2SO4 and evaporated under vacuum to afford oleic acid NHS ester as a white solid (1.13 g). To a solution of aminohexyl-VB12 (100 mg) in DMF (1 mL) was added triethylamine (3 drops), followed by a solution of oleic acid NHS ester (35.3 mg) in DMF (1 mL). The solution was stirred for 4 hours, then added to ethyl acetate (40 mL) and the resulting precipitate was centrifuged, washed with ethyl acetate (2×20 mL) and dried under high vacuum to afford oleamidohexyl-VB12 (VB12-Oleamide) as a red solid (108 mg).

### Example 19

### Synthesis of VB12-Stearamide

To a suspension of stearic acid (1.0 g) and NHS (445 mg) in dichloromethane (50 mL) was added EDAC (740 mg) and the mixture was stirred for 24 hours at room temperature. The solution was washed with ice-cold water (3×50 mL), dried over Na2SO4 and evaporated under vacuum to afford stearic acid NHS ester as a white solid (1.2 g). To a solution of aminohexyl-VB12 (100 mg) in DMF (5 mL) was added triethylamine (5 drops), followed by a solution of stearic acid NHS ester (35.5 mg) in DMF (2 mL). The solution was stirred for 5 hours, then added to ethyl acetate (50 mL) and the resulting precipitate was centrifuged, washed with ethyl acetate (2×25 mL) and dried under high vacuum to afford stearamidohexyl-VB12 (VB12-Stearamide) as a red solid (102 mg).

### Example 20

### Synthesis of VB12-Coated PLGA Nanoparticles Containing Insulin

A 20 mL vial was charged with recombinant human insulin (10.0 mg), PLGA (RG 502H, 150 mg), and a solvent system of acetone (12.0 mL) and 10 mM HCl (1.85 mL). This was mixed on an orbital shaker for 40 min. The resulting solution was added quickly to rapidly stirring 8.3 mg/mL PVA solution (Mowiol 4-88, 180 mL) immediately becoming turbid. The white suspension was stirred for 1 h then centrifuged at 10,500 rpm for 20 min at 4° C. and the supernatant decanted. The pellet was washed with deionized water (2×40 mL) then re-suspended in 30 mL deionized water. To this stirring suspension was added 1 mg/mL VB12-Oleamide solution (250 μL in EtOH), the mixture stirred for 22 h and then centrifuged at 10,500 rpm for 20 min at 4° C. The supernatant was decanted and the pellet was lyophilized to yield 92.6 mg of pink nanoparticles; Z average=219 nm, PDI=0.058; zeta potential=−34.2 mV.

### Example 21

### Synthesis of VB12-Coated PLGA-HP55 Nanoparticles Containing Insulin

A 20 mL vial was charged with recombinant human insulin (10.0 mg), PLGA (RG 502H, 150 mg), HP-55 (75 mg) and a solvent system of acetone (12.0 mL) and 10 mM HCl (1.85 mL). This was mixed on an orbital shaker for 40 min. The resulting solution was added quickly to rapidly stirring 8.3 mg/mL PVA solution (Mowiol 4-88, 180 mL) immediately becoming very turbid. The white suspension was stirred for 1 h then centrifuged at 10,500 rpm for 20 min at 4° C. and the supernatant decanted. The pellet was washed with deionized water (2×40 mL) then re-suspended in 30 mL deionized water. To this stirring suspension was added 1 mg/mL VB12-Oleamide solution (250 μL in EtOH), the mixture stirred for 22 h and then centrifuged at 10,500 rpm for 20 min at 4° C. The supernatant was decanted and the pellet was lyophilized to yield 151 mg of pink nanoparticles; Z-average=292 nm, PDI=0.063; Zeta potential=−55.8 mV.

### Example 22

### Synthesis of VB12-Coated PLGA-HP55 Nanoparticles Containing Insulin

A 7 mL vial was charged with human recombinant insulin (2.0 mg), RG 502H PLGA (30.0 mg) and HP-55 (15 mg). A mixed solvent system of 2.4 mL acetone and 0.4 mL 10 mM HCl was then added with rapid shaking for 20 min to produce a clear solution. 500 μL of 5.0 mg/mL VB12-Oleamide solution in EtOH was added to 30 mL of 10 mg/mL PVA solution (Mowiol 4-88), and the insulin/PLGA/HP-55 solution added dropwise to this over the course of 3 min with rapid stirring, forming a turbid pink suspension. This suspension was stirred for 1 h and then centrifuged at 10500 rpm for 20 min at 4° C. and the supernatant decanted. The pellet was washed with deionized water (2×15 mL) and lyophilized to yield 31.2 mg of pink nanoparticles; Z-average=280 nm, PDI=0.138; Zeta potential=−44.9 mV.

### Example 23

### Synthesis of VB12/Pluronic-Coated PLGA Nanoparticles Containing siRNA

100 μL of 10 mg/mL siRNA solution in pH 7.5 TE (Tris-EDTA) buffer was briefly combined with 200 μL of 20 mg/mL acetylated Bovine Serum Albumin solution, also in pH 7.5 TE buffer. 750 μL of RG 502H PLGA in dichloromethane was then added and the mixture was sonicated for 1 min. To the resulting white emulsion was added 2 mL of 20 mg/mL PVA solution (10 k MW, 80% hydrolyzed), with sonication for an additional 1 min. This emulsion was then added to 12 mL of 20 mg/mL PVA solution, and the mixture stirred in an uncapped 20 mL vial for 1.5 h. It was then centrifuged at 10,500 rpm for 20 min at 4° C. and the supernatant decanted. The pellet was washed with deionized water (2×5 mL) and then re-suspended in 3 mL deionized water. To this suspension was added 100 μL of 1 mg/mL VB12-Stearamide solution in EtOH and the mixture was shaken for 5 h. 300 μL of 10 mg/mL Pluronic F68 was then added with shaking for 16 h. The mixture was again centrifuged at 10,500 rpm for 20 min at 4° C., the supernatant decanted, the pellet was washed with 3 mL deionized water and finally lyophilized to yield 14.3 mg of pink nanoparticles; Z-average=198 nm, PDI=0.059; zeta potential=39.2 mV.

### Example 24

### Synthesis of VB12-Coated PLGA-TMC Nanoparticles Containing siRNA

100 μL of 10 mg/mL TMC hexafluorophosphate solution in DMSO and 750 μL of 40 mg/mL RG 502H PLGA solution in dichloromethane were briefly combined and 300 μL of 3.33 mg/mL siRNA solution in pH 7.5 TE buffer was added. The mixture was sonicated for 1 min and then 2 mL of 20 mg/mL PVA solution (10 k MW, 80% hydrolyzed) added to the emulsion with sonication for an additional 1 min. This emulsion was subsequently added to 12 mL of 20 mg/mL PVA solution, and the mixture stirred in an uncapped 20 mL vial for 1.5 h. It was then centrifuged at 10,500 rpm for 20 min at 4° C. and the supernatant decanted. The pellet was washed with deionized water (2×5 mL) and then lyophilized to yield 17.3 mg of white nanoparticles; Z-average=206 nm, PDI=0.036; zeta potential=−34.1 mV. A 7 mL vial was charged with 8.2 mg of dried nanoparticles and 2 mL deionized water added with shaking for 10 min. To the resulting white suspension was added 100 μL of 1 mg/mL VB12-Oleamide solution in EtOH and the mixture was shaken for 3 h, then centrifuged at 13,000 rpm for 30 min. The supernatant was decanted and the pellet lyophilized to yield 9.0 mg of pink nanoparticles; Z-average=193 nm, PDI=0.057; zeta potential=−35.6 mV.

### Example 25

### Synthesis of VB12-TMC siRNA Polyelectrolyte Complex Nanoparticles

1.0 mL of 1.0 mg/mL siRNA solution in 1.5% dextrose/pH 4 100 mM acetate buffer was added to 1.0 mL of 1.5 mg/mL VB12-TMC solution in 1.5% dextrose/pH 4 100 mM acetate buffer and stirred for 10 min to form a clear pink solution; Z-average=126 nm, PDI=0.181; Zeta potential=22.8 mV.

### Example 26

### Synthesis of VB12-Coated PLGA Nanoparticles Containing Leuprolide

100 μL of 10 mg/mL leuprolide solution in pH 7.5 TE buffer was combined with 200 μL of 20 mg/mL acetylated Bovine Serum Albumin solution, also in pH 7.5 TE buffer and shaken for 3 min. 750 μL of RG 502H PLGA in dichloromethane was then added and the mixture was sonicated for 1 min. To the resulting white emulsion was added 2 mL of 20 mg/mL PVA solution (10 k MW, 80% hydrolyzed), with sonication for an additional 1 min. This emulsion was then added to 12 mL of 20 mg/mL PVA solution, and the mixture stirred in an uncapped 20 mL vial for 1.5 h. It was then centrifuged at 10,500 rpm for 20 min at 4° C. and the supernatant decanted. The pellet was washed with deionized water (2×5 mL) and lyophilized to yield 17.8 mg of nanoparticles; Z-average=205 nm, PDI=0.028; Zeta potential=−36.2 mV. A 7 mL vial was charged with 4.2 mg of dried nanoparticles and 1 mL deionized water added with shaking for 15 min. To the resulting white suspension was added 50 μL of 1 mg/mL VB12-Stearamide solution in EtOH and the mixture was shaken for 48 h, then centrifuged at 13,000 rpm for 30 min. The supernatant was decanted and the pellet lyophilized to yield 5.0 mg of pink nanoparticles; Z-average=204 nm, PDI=0.021; zeta potential=−35.0 mV.

### Example 27

### Synthesis of VB12-Coated PLGA Nanoparticles Containing Exenatide

100 μL of 10 mg/mL exenatide solution in pH 7.5 TE buffer was briefly combined with 200 μL of 20 mg/mL acetylated Bovine Serum Albumin solution, also in pH 7.5 TE buffer. 750 μL of RG 502H PLGA in dichloromethane was then added and the mixture was sonicated for 1 min. To the resulting white emulsion was added 2 mL of 20 mg/mL PVA solution (10 k MW, 80% hydrolyzed), with sonication for an additional 1 min. This emulsion was then added to 12 mL of 20 mg/mL PVA solution, and the mixture stirred in an uncapped 20 mL vial for 1.5 h. It was then centrifuged at 10,500 rpm for 20 min at 4° C. and the supernatant decanted. The pellet was washed with deionized water (2×5 mL) and lyophilized to yield 16.3 mg of nanoparticles; Z-average=210 nm, PDI=0.092; Zeta potential=−35.9 mV. A 7 mL vial was charged with 9.0 mg of dried nanoparticles and 2 mL deionized water added with shaking for 15 min. To the resulting white suspension was added 100 μL of 1 mg/mL VB12-Stearamide solution in EtOH and the mixture was shaken for 48 h, then centrifuged at 13,000 rpm for 30 min. The supernatant was decanted and the pellet lyophilized to yield 10.2 mg of pink nanoparticles; Z-average=266 nm, PDI=0.196; zeta potential=−33.3 mV.

### Example 28

### Synthesis of VB12-PEG-PLGA

Vitamin B12 (1.65 g) was added to dry DMSO (30 mL) with rapid stirring and the deep purple solution was stirred for 30 min. 4 A Molecular Sieves were added and stirring continued for 20 min. Carbonyl-1,1-ditriazole (CDT; 1.0 g) was added and the mixture stirred for 3 hr., then poured into ethyl acetate (150 mL). The precipitate was centrifuged, washed with ethyl acetate and dried under high vacuum to afford VB12-CT (2.06 g). A solution of monotrityl diaminotriethyleneglycol (400 mg) in dry DMSO (4 mL) was added to a solution of VB12-CT (600 mg) in dry DMSO (4 mL) and the mixture stirred for 20 hr. Triethylamine (0.1 mL) was added and the mixture stirred for 2 hr. then poured into stirring ethyl acetate (50 mL). The resulting red suspension was centrifuged then washed with ethyl acetate (3×50 mL) and dried under high vacuum to afford tritylaminotriethyleneglycolamido VB12 (690 mg) as a dark red powder. Ttritylamino-triethyleneglycolamido VB12 (660 mg) was added slowly to a mixture of trifluoroacetic acid (5 mL) and dichloromethane (15 mL) and the mixture stirred for 2 hr., then added to a 1:2 mixture of ethyl acetate and heptane. The precipitate was centrifuged, washed with ethyl acetate and dried under high vacuum to afford aminotriethyleneglycol-amido VB12 (ATG-VB12; 480 mg) as a dark red powder.

To a solution of poly(lactic-co-glycolic acid) (PLGA RG 502H; 100 mg) and NHS (60 mg) in dichloromethane (20 mL) was added EDAC (100 mg) and the mixture was stirred for 20 hours at room temperature. The solution was evaporated to ˜5 mL, then added to diethyl ether (20 mL). The resulting solid was washed with ether and vacuum dried to afford PLGA NHS ester as a white solid (180 mg). To a solution of PLGA NHS ester (170 mg) in dichloromethane (5 mL) was added H2N-PEG1k-CO2H (25 mg), followed by a triethylamine (50 μL). The solution was stirred for 18 hours, the solvent evaporated and the residue dissolved in dichloromethane (10 mL). The solution was washed with water (3×10 mL), the solvent was evaporated and the product dried under high vacuum to afford PLGA-PEG1kCO2H (65 mg). PLGA-PEG1kCO2H (65 mg) was dissolved in DMF (5 mL) and N,N,N′,N′-tetramethyl-O-(1H-benzotriazol-1-yl)uronium hexafluorophosphate (HBTU; 5.7 mg) and diisopropylethylamine (0.1 mL) were added and the mixture was stirred for 5 min. A solution of aminotriethyleneglycolamido-VB12 (ATG-VB12; 24 mg) in DMF (2 mL) was added and the reaction stirred for 16 hours, then poured into diethyl ether (35 mL). The solid was separated by centrifugation and dissolved in dichloromethane (30 mL), washed with water (3×30 mL) and dried over Na2SO4. The solvent was evaporated to ˜2 mL and added to ether. The precipitate was separated by centrifugation and the product dried under high vacuum to afford PLGA-PEG lk amidotriethyleneglycolamido-VB12 (VB12 PEG PLGA) (30 mg) as a pale red solid. Cobalt analysis by ICP revealed the product contained 8623.5 ppm of cobalt, which corresponds to 19.8% w/w of VB12.

### Example 29

### Synthesis of VB12-PEG-DSG

A mixture of 1,2-O-distearyl-sn-glycerol (DSG; 500 mg), N,N′-disuccinimidyl carbonate (DSC; 322 mg) and triethylamine (0.4 mL) in dichloromethane (15 mL) was stirred for 14 hr. The solution was washed with water (3×15 mL), dried over Na2SO4 and the solvent evaporated to afford DSG-CONHS (544 mg). To an ice-cooled solution of DSG-CONHS (431 mg) and H2N-PEG2k-CO2H (900 mg) in dichloromethane (15 mL) was added pyridine (2 mL). The solution was stirred at room temperature for 24 hr. then concentrated under vacuum. The residue was subjected to preparative chromatography on silica gel, eluting with ethyl acetate/hexane then MeOH/CH2Cl2 to afford DSG-PEG2kCO2H (941 mg) as a white solid. To a solution of DSG-PEG2kCO2H (270 mg) and NHS (100 mg) in dichloromethane (20 mL) was added EDAC (33 mg) and the mixture was stirred overnight at room temperature. A further portion of EDAC (128 mg) was added and stirred for 2 hr., followed by further portions of EDAC (50 mg) and NHS (50 mg) and further stirring overnight. The solution was diluted with dichloromethane (70 mL) then washed with water and brine (2×25 mL each), dried over Na2SO4, the solvent evaporated and the residue dried under high vacuum to afford DSG-PEG2kCO2H NHS ester (275 mg).

To a solution of aminohexylamido-VB12 (AH-VB12; 175.4 mg) and triethylamine (0.3 mL) in DMF (5 mL) was added a solution of DSG-PEG2kCO2H NHS ester (270 mg) in DMF (5 mL) and the reaction stirred for 17 hours, then poured into ethyl acetate (100 mL). The solid was separated by centrifugation, washed with ether and dried under high vacuum to afford DSG-CO-PEG2k carboxyamidohexylamido-VB12 (VB12 PEG2k DSG) (353 mg) as a pale red solid.

Although several embodiments of the invention are described herein in detail, it will be understood by those skilled in the art that variations may be made thereto without departing from the spirit of the invention or the scope of the appended claims.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A nanoparticle comprising a therapeutic agent encapsulated by one or more polymer(s) to which vitamin B12 or a derivative thereof is attached to the at least one or more polymer(s) via a linker group.

2. A micelle comprising a therapeutic agent encapsulated by the micelle and vitamin B12 or a derivative thereof attached to the micelle as a targeting agent.

3. The nanoparticle of claim 1, wherein the one or more polymers is selected from a synthetic polymer, a semi-synthetic polymer, a natural polymer or a polymer capable of forming a polyelectrolyte complex (PEC).

4. The micelle of claim 2, wherein the one or more polymers is selected from a synthetic polymer, a semi-synthetic polymer, a natural polymer or a polymer capable of forming a polyelectrolyte complex (PEC).

5. The nanoparticle of claim 1, wherein the vitamin B12 or a derivative thereof is one or more of VB12-5′-O-carboxytriazole, VB12-5′-O-carboxylmidazole, VB12-5′-O-carboxyamido-C2-C20-alkylamines, VB12-5′-O-carboxyamido-oligoethyleneoxyamines, and dicarboxylic acid derivatives of the aforementioned compounds.

6. The micelle of claim 2, wherein the vitamin B12 or a derivative thereof is one or more of VB12-5′-O-carboxytriazole, VB12-5′-O-carboxylmidazole, VB12-5′-O-carboxyamido-C2-C20-alkylamines, VB12-5′-O-carboxyamido-oligoethyleneoxyamines, and dicarboxylic acid derivatives of the aforementioned compounds.

7. The nanoparticle of claim 1, further comprising a polymer coating encompassing the nanoparticle.

8. The micelle of claim 2, further comprising a liposome encapsulating the micelle.

9. The nanoparticle of claim 7, further comprising a vitamin B12 or a derivative thereof or other targeting agent attached via a linking group to the one or more polymer(s) of the coating.

10. The liposome of claim 8, further comprising a vitamin B12 or a derivative thereof or other targeting agent attached via a linking group to the one or more polymer(s) of the coating.

11. The nanoparticle of claim 1, further comprising a targeting agent other than vitamin B12 or a derivative thereof attached to the nanoparticle.

12. The micelle of claim 2, further comprising a targeting agent other than vitamin B12 or a derivative thereof attached to the micelle.

13. The liposome of claim 8, further comprising a targeting agent other than vitamin B12 or a derivative thereof attached to the liposome.

14. A composition comprising a nanoparticle of claim 1 and a carrier.

15. A composition comprising the micelle of claim 2 and a carrier.

16. A method for delivering a therapeutic agent in vivo, comprising administering to a subject an effective amount of a nanoparticle of claim 1, thereby delivering the therapeutic agent.

17. A method for delivering a therapeutic agent in vivo, comprising administering to a subject an effective amount of a micelle of claim 2, thereby delivering the therapeutic agent.

18. A method for delivering a therapeutic agent in vivo, comprising administering to a subject an effective amount of a liposome of claim 8, thereby delivering the therapeutic agent.

19. A method for preparing a nanoparticle composition comprising admixing a therapeutic agent and at least one polymer to which vitamin B12 or a derivative thereof is attached by a linker group in a suitable solvent and optionally, wherein the ratio of the polymer to the therapeutic agent is in a range selected from the group of 1 to 15%, 1 to 40%, 5 to 50%, 5 to 40%, 5 to 30%, 10 to 35%, or 10 to 30%.

20. The method of claim 19, further comprising admixing a second targeting agent other than vitamin B12 or a derivative thereof in the suitable solvent.

21. The method of claim 19, further comprising modifying the nanoparticles to effect cross-linking of components of the nanoparticles wherein the components comprise metal ions, small molecules having at least two positively charged groups or two negatively-charged groups, or small molecules that react to form at least two covalent bonds.

22. The method of claim 19, further comprising isolating, purifying, and/or drying the nanoparticles from the solvent.

23. A nanoparticle prepared by the method of claim 19.
